(19) United States
(12) Patent Application Publication
(10) Pub. No.: US 2008/0113016 A1
(43) Pub. Date: May 15, 2008

(54) Method for Prevention or Treatment of Inflammatory Disease

(76) Inventors: Gary Steven Firestein, Del Mar, CA (US); David Louis Boyle, Santee, CA (US); Linda Sue Sorkin, Encinitas, CA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 11/572,797

(22) PCT Filed: Jul. 25, 2005
(86) PCT No.: PCT/US05/26312

§ 371 Date: Jun. 21, 2007

Related U.S. Application Data
(60) Provisional application No. 60/591,327, filed on Jul. 26, 2004.
(60) Provisional application No. 60/606,580, filed on Sep. 1, 2004.

(51) Int. Cl. A61K 9/50; A61K 9/127; A61K 31/47

(52) U.S. Cl. 424/450; 424/490; 514/310; 977/906

(57) ABSTRACT

Methods are provided for prevention or treatment of inflammatory disease in a mammal by administering an inhibitor of mitogen activated (MAP) kinase system to the mammal in a therapeutic amount to the mammal in need thereof. The MAP kinase inhibitor is targeted to the central nervous system of the mammal. Methods are further provided for prevention or treatment of inflammatory disease in a mammal by administering an antagonist of TNF-α to the mammal in a therapeutic amount to the mammal in need thereof. The TNF-α antagonist is targeted to the central nervous system of the mammal.

### CROSS REFERENCE TO RELATED APPLICATION

This application is related to U.S. Application No. 60/591,327, filed Jul. 26, 2004, and U.S. Application No. 60/606,580, filed Sep. 1, 2004, the disclosures of which are incorporated herein by reference in their entirety.

### GOVERNMENT RIGHTS

The work leading to the disclosed inventions was funded in whole or in part with Federal funds from the National Institute of Health under Grant No. AR47825. Accordingly, the U.S. Government has rights in these inventions.

### FIELD

The invention relates to methods for preventing or treating inflammatory disease in a mammal comprising administering an inhibitor of mitogen activated (MAP) kinase targeted to the central nervous system of the mammal in a therapeutic amount to the mammal in need thereof. The invention further relates to methods for preventing or treating inflammatory disease in a mammal comprising administering a TNF-α antagonist to the central nervous system of the mammal in a therapeutic amount to the mammal in need thereof.

### BACKGROUND

MAP kinases play a role in innate immunity and host defense by regulating cytokines, like IL-1 and TNF-α, as well as enzymes involved in tissue remodeling, like matrix metalloproteinases (MMPs). IL-1 and TNFα have been shown to be central players in the pathological processes underlying many chronic inflammatory and autoimmune diseases. IL-1 is implicated in mediating or exacerbating diseases such as rheumatoid arthritis (Arend, W. P. Arthritis & Rheumatism 38: 151-160, 1995), osteoarthritis, bone resorption, toxic shock syndrome, tuberculosis, atherosclerosis, diabetes, Hodgkin's disease, (Benharroch, D.; et al., Euro. Cytokine Network 7: 51-57), and Alzheimer's disease. Excessive or unregulated TNF production has been implicated in mediating or exacerbating diseases such as rheumatoid arthritis, (Maini, R. N.; et al., APMIS 105: 257-263, 1997; Feldmann, M., J. of the Royal College of Physicians of London 30: 560-570, 1996); Lorenz, H. M.; et al., J. of Immunology 156: 1646-1653, 1996); osteoarthritis, spondylitis, sepsis, septic shock, (Abraham, E.; et al., JAMA. 277:1531-1538, 1997); adult respiratory distress syndrome, asthma (Shah, A.; et al., Clin. & Exp. Allergy 1038-1044, 1995; Lassalle, P., et al., Clin. & Exp. Immunol. 94: 105-110, 1993), bone resorption diseases, fever, (Cooper, A. L., et al., Am. J. of Physiology 267: 1431-1436), encephalomyelitis, demyelination, (Klindert, W. E.; et al., J. of Neuroimmunol. 72: 163-168, 1997), and periodontal diseases.

Clinical trials with IL-1 and TNF receptor antagonists have shown that blocking the ability of these cytokines to signal through their receptors leads to significant improvement in inflammatory diseases in humans. Therefore, modulation of these inflammatory cytokines has positive therapeutic outcomes. It has also been shown that p38 MAP kinase plays a role in the translational control of TNF and IL-1 and is also involved in the biochemical signaling of these molecules, Lee, J. C., et al., Nature. 372: 739-46, 1994. Compounds that inhibit p38 MAP kinase are effective in inhibiting bone resorption, inflammation, and other immune and inflammation-based pathologies. Side effects of therapeutic treatment utilizing MAP kinase inhibitors or TNF-α antagonists include systemic toxicity in the liver and other organs of a patient.

Interest in signal transduction pathways that regulate anti-inflammatory neural networks was stimulated by the demonstration that peripheral nerve injury activates MAP kinases in the spinal cord and plays a role in nociception. Svensson, et al., Neuroreport, 14: 1153-1157, 2003; Svensson et al., J. Neurochem 86: 1534-1544, 2003. The MAP kinase system is a complex, interrelated signal transduction system that integrates extracellular stresses and induces an appropriate cellular response (see below for a simplified outline of the MAPK cascade). The three major MAP kinase families, c-Jun-N-terminal kinase (JNK), extracellular regulating kinase (ERK) and p38, differ in their substrate specificity and subsequent responses to stress depending on the cell type and the environmental influences. Robinson, et al., Curr. Opin. Cell. Biol., 9: 180-185, 1997. A fourth MAPK pathway involving ERK5 has also been described. The MAP kinases regulate various genes via both transcriptional and post-transcriptional mechanisms. Seger, et al., RFaseb. J., 9: 726-735, 1995; Fanger, et al., Curr. Opin. Genet. Dev., 7: 67-76, 1997. Upstream MAP kinase kinases (MAPKK) serve as regulators of MAP kinase activity by phosphorylating specific threonine and tyrosine residues. MAPKKs are, in turn, regulated, by MAPKK kinases (MAPKKK or MAP3K). Hibi, et al., Genes Dev., 7: 2149-2160, 1993.

MAP kinases play a key role in innate immunity and host defense by regulating cytokines, like IL-1 and TNF-α, as well as enzymes involved in tissue remodeling, like matrix metalloproteinases (MMPs). Surprisingly, the same pathways are activated in spinal cord neurons, microglia, and dorsal root ganglion (DRG) neurons when peripheral nerves are injured by a variety of noxious stimuli. Jin, et al., J. Neurosci., 23: 4017-4022, 2003. For instance, phosphorylated p38 (P-p38) increases in DRG neurons within a few hours after spinal nerve ligation (SNL), a standard model of chronic pain. Schäfers, et al., Neurosci. Lett., 347: 179-182, 2003. p38 blockade by spinal administration of a p38 inhibitor substantially reduces SNL-induced mechanical allodynia. After unilateral chronic constriction injury (CCI) to the mid-sciatic nerve (another standard pain model), P-p38 in small DRG neurons is elevated up to 2 weeks after injury. Kim, et al., Neuroreport, 13: 2483-2486, 2002.

Prominent increases in laminae I-IV staining for P-p38 is observed 1 and 2 weeks following CCI and sciatic nerve transection and as early as 24-48 hrs after root avulsion and spinal nerve axotomy. Nomura, et al., Brain Res., 893: 84-94, 2001. The vast majority of this increase occurs in the microglia, which are also activated by the various nerve injuries. Etanercept, the TNF-α antagonist, blocks the increase in the spinal cord P-p38 when administered intrathecally before SNL. Schäfers, et al., J. Neurosci., 23: 2517-2521, 2003. These observations suggest that TNF-α might trigger p38 phosphorylation (rather than the reverse) in neurons and microglia. Hence, nerve injury and cytokine production, which can also occur in tissue damage or inflammation, could play a key role in the DRG and spinal cord p38 activation.

With regard to the other members of the MAPK family, comparatively few studies have examined JNK following nerve injury. JNK activity increases after sciatic transection 1 and 4 cm from the DRG in rats. Kenney, et al., J. Neurosci., 18: 1318-1328, 1998. Latency is 30 min for proximal lesions and 3 hr for distal lesion, implying that retrograde transport is involved. Following partial spinal nerve ligation, P-JNK increases after 1 and 2 weeks in both the L4-5 spinal dorsal horn and the nucleus gracilis. Spinal P-JNK co-localizes with GFAP, indicating that it is in reactive astrocytes. Ma, et al., Pain, 99: 175-184, 2002. Several studies have examined ERK activation in DRG neurons resulting from acute pain, C-fiber stimulation, or inflammation. In spinal dorsal horn and in nucleus gracilus, P-ERK is localized to reactive astrocytes 1 and 2 weeks following partial nerve ligation. CCI causes increased P-ERK in the lumbar spinal cord; intrathecal administration of a selective MEK inhibitor blocks ERK phosphorylation and reduces the CCI-associated allodynia. Ciruela, et al., Br. J. Pharmacol., 138: 751-756, 2003.

Data on activation of MAP kinases in the CNS due to inflammation are less clear. Carrageenan injection into rat footpads induces a 5-fold increase in spinal ERK phosphorylation. Galan, et al., Neurosci. Lett., 322: 37-40, 2002. A similar increase in ERK activation occurs in an inflammatory pain model induced by injection of complete Freund's adjuvant (CFA) into rat paws. Obata, et al., J. Neurosci., 23: 4117-4126, 2003. Immunohistochemical studies show that both neurons and glial cells in the spinal cord contain the activated kinase. ERK activation after formalin injection appears to require metabotropic glutamate receptors rather than NMDA receptors. Karim, et al., J. Neurosci., 21: 3771-3779, 2001. Formalin or zymosan injection into the hind paws of rats also activates glial cells with release of spinal IL-1. Sweitzer, et al., Brain Res., 829: 209-221, 1999. One study suggests that p38 can be activated in spinal cords as a result of peripheral inflammation although the stimulus, formalin, is primarily a mediator of nerve injury and spinal sensitization rather than inflammatory pathways. Kim, et al., Neuroreport., 13: 2483-2486, 2002. Spinal cord neurons and microglial cells as well as DRGs stain positively for P-p38 in that model.

In addition to MAP kinase activation, cytokines can be released in the CNS after nerve injury and inflammation. Endogenous TNFα levels increase in axons and in cell bodies of both injured and adjacent uninjured DRG neurons (Schäfers, et al., J. Neurosci., 22: 536-545, 2002) and in the spinal cord. Sweitzer, et al., Brain Res., 829: 209-221, 1999. Some of the TNFα might be transported from the peripheral site of injury. Activated Schwann cells at the injury site produce TNFα (Shubayev, et al., J. Neuroimmunol., 114: 48-56, 2001) as well as IL-1. Shamash, et al., J. Neurosci., 22: 3052-3060, 2002. TNFα released at the site of CCI injury binds to its receptors (TNFR1 and TNFR2) and is then retrograde transported towards the DRG at an estimated rate of 300 mm/day. TNFα, in nerve injured, but not in control animals, is transported from sensory afferent fibers into the neuropil of the dorsal horn, into small neurons, and perhaps glia. Invading macrophages represent another significant source of pro-inflammatory cytokines in the DRG of nerve-injured animals, (Hu, et al., Neuroscience, 112: 23-38, 2002) as they synthesize both TNFα and IL-1. Nerve injury induces prominent increases in spinal cord TNF and IL-1. Hashizume, et al., Spine, 25: 1206-1217, 2000. The most abundant source of these cytokines is probably astrocytes and microglia, (Hanisch, et al., Glia, 40: 140-155, 2002) rather than transport from the periphery.

A need exists in the art for an effective treatment for arthritis and inflammation by administration of compounds that inhibit inflammation pathways, thereby providing a means of combating diseases while minimizing toxic side effects of such treatment.

### SUMMARY

The present invention provides a method for preventing or treating inflammatory disease or arthritis in a mammal comprising administering an inhibitor of mitogen activated protein (MAP) kinase targeted to the central nervous system of the mammal in a therapeutic amount to the mammal in need thereof. The method targets therapeutic compounds to blockade key signal transduction enzymes in the CNS, for example, MAP kinases or p38 MAP kinases. The present invention further provides a method for preventing or treating inflammatory disease or arthritis in a mammal comprising administering a TNF-α antagonist targeted to the central nervous system of the mammal in a therapeutic amount to the mammal in need thereof.

A method for preventing or treating inflammatory disease in a mammal is provided comprising administering an inhibitor of mitogen activated (MAP) kinase targeted to the central nervous system of said mammal in a therapeutic amount to said mammal in need thereof. The MAP kinase includes, but is not limited to, p38 MAP kinase, p38a MAP kinase, and p3813 MAP kinase. The MAP kinase can be, for example, JNK or MEK1/2. In one aspect, the inflammatory disease is peripheral inflammation, acute inflammation, chronic inflammation, arthritis, or rheumatoid arthritis. In a detailed aspect, the inflammatory disease is bone resorption, graft vs. host reaction, atherosclerosis, arthritis, osteoarthritis, rheumatoid arthritis, gout, psoriasis, topical inflammatory disorder state, adult respiratory distress syndrome, asthma, chronic pulmonary inflammatory disorder, cardiac reperfusion injury, renal reperfusion injury, thrombus, glomerulonephritis, Crohn's disorder, ulcerative colitis, inflammatory bowel disorder, or cachexia.

The MAP kinase inhibitor can be, for example, an antisense oligonucleotide to p38 MAP kinase, p38α MAP kinase, or p38β MAP kinase. The MAP kinase inhibitor can be, for example, an interfering RNA to p38 MAP kinase, p38α MAP kinase, or p38β MAP kinase. In a detailed aspect, the p 38 MAP kinase inhibitor is SB023580. In a further detailed aspect, the MAP kinase inhibitor is SP600125 or PD98059.

In a further embodiment, the MAP kinase inhibitor is administered intrathecally, intramedullarly, intracerebrally, intracerebroventricularly, intracranially, epidurally, intraspinally, or intraparietally. In one aspect, the MAP kinase inhibitor crosses the blood-brain barrier of the mammal. In a further aspect, the MAP kinase inhibitor is administered intranasally to the mammal.

In a further embodiment, the MAP kinase inhibitor is administered systemically. The MAP kinase inhibitor can be administered, for example, intravenously, parenterally, subcutaneously, intramuscularly, ophthalmicly, intraventricularly, intraperitoneally, orally, or topically, to said mammal. In a further aspect, the MAP kinase inhibitor is administered in an encapsulated form in a lipophilic compound or liposome.

The MAP kinase inhibitor can be administered, for example, intrathecally into the cerebrospinal fluid of the subject, in an encapsulated form at an entry region. In a further aspect, the entry region is not a lumbar region. In a further aspect, MAP kinase inhibitor is administered intrathecally to a sacral region of the central nervous system of the subject. In a further aspect, the MAP kinase inhibitor is encapsulated in a polymer. The polymer can be, for example, a naturally derived polymer, albumin, alginate, cellulose derivatives, collagen, fibrin, gelatin, or polysaccharide. The polymer can be, for example, a synthetic polymer, polyester, polylactic acid, polyglycolic acid, polylactic-co-glycolic acid, polyethylene glycol, poloxomer block copolymer, or polyanhydride. In a detailed aspect, the therapeutic agent in an encapsulated form is introduced into a cerebral ventricle. In a further aspect, an encapsulated form of the therapeutic agent is introduced into the cisterna magna. In a further aspect, an encapsulated form of the therapeutic agent is introduced into the lumbar region.

A method of treating, reducing, or preventing inflammation is provided comprising contacting the periphery of a mammal with a compound that decreases the enzymatic activity or phosphorylation level of a MAP kinase in the central nervous system of the mammal, in an amount sufficient to treat, reduce, or prevent inflammation.

In a further embodiment, the MAP kinase inhibitor is administered intrathecally, intramedullarly, intracerebrally, intracerebroventricularly, intracranially, epidurally, intraspinally, or intraparietally. In one aspect, the MAP kinase inhibitor crosses the blood-brain barrier of the mammal. In a further aspect, the MAP kinase inhibitor is administered intranasally to the mammal.

In a further embodiment, the MAP kinase inhibitor is administered systemically. The MAP kinase inhibitor can be administered, for example, intravenously, parenterally, subcutaneously, intramuscularly, ophthalmicly, intraventricularly, intraperitoneally, orally, or topically, to said mammal. In a further aspect, the MAP kinase inhibitor is administered in an encapsulated form in a lipophilic compound or liposome. In a further aspect, the MAP kinase inhibitor is encapsulated in a polymer.

A method for preventing or treating inflammatory disease in a mammal is provided comprising administering an antagonist of TNF-α targeted to the central nervous system of said mammal in a therapeutic amount to said mammal in need thereof. In one aspect, the inflammatory disease is peripheral inflammation, acute inflammation, chronic inflammation, arthritis, or rheumatoid arthritis. In a detailed aspect, the inflammatory disease is bone resorption, graft vs. host reaction, atherosclerosis, arthritis, osteoarthritis, rheumatoid arthritis, gout, psoriasis, topical inflammatory disorder state, adult respiratory distress syndrome, asthma, chronic pulmonary inflammatory disorder, cardiac reperfusion injury, renal reperfusion injury, thrombus, glomerulonephritis, Crohn's disorder, ulcerative colitis, inflammatory bowel disorder, or cachexia. The TNF-α antagonist can be, for example, an antisense oligonucleotide or an interfering RNA. The TNF-α antagonist can be, for example, etanercept, infliximab, or adalimumab.

In a further embodiment, the TNF-α antagonist is administered intrathecally, intramedullarly, intracerebrally, intracerebroventricularly, intracranially, epidurally, intraspinally, or intraparietally. In one aspect, the TNF-α antagonist crosses the blood-brain barrier of the mammal. In a further aspect, the TNF-α antagonist is administered intranasally to the mammal.

In a further embodiment, the TNF-α antagonist is administered systemically. The TNF-α antagonist can be administered, for example, intravenously, parenterally, subcutaneously, intramuscularly, ophthalmicly, intraventricularly, intraperitoneally, orally, or topically, to said mammal. In a further aspect, the TNF-α antagonist is administered in an encapsulated form in a lipophilic compound or liposome.

The TNF-α antagonist can be administered, for example, intrathecally into the cerebrospinal fluid of the subject, in an encapsulated form at an entry region. In a further aspect, the entry region is not a lumbar region. In a further aspect, TNF-α antagonist is administered intrathecally to a sacral region of the central nervous system of the subject. In a further aspect, the TNF-α antagonist is encapsulated in a polymer. The polymer can be, for example, a naturally derived polymer, albumin, alginate, cellulose derivatives, collagen, fibrin, gelatin, or polysaccharide. The polymer can be, for example, a synthetic polymer, polyester, polylactic acid, polyglycolic acid, polylactic-co-glycolic acid, polyethylene glycol, poloxomer block copolymer, or polyanhydride. In a detailed aspect, the therapeutic agent in an encapsulated form is introduced into a cerebral ventricle. In a further aspect, an encapsulated form of the therapeutic agent is introduced into the cisterna magna. In a further aspect, an encapsulated form of the therapeutic agent is introduced into the lumbar region.

A method of treating, reducing, or preventing inflammation is provided comprising contacting the periphery of a mammal with a compound that decreases the activity of TNF-α in the central nervous system of the mammal, in an amount sufficient to treat, reduce, or prevent inflammation.

In a further embodiment, the TNF-α antagonist is administered intrathecally, intramedullarly, intracerebrally, intracerebroventricularly, intracranially, epidurally, intraspinally, or intraparietally. In one aspect, the TNF-α antagonist crosses the blood-brain barrier of the mammal. In a further aspect, the TNF-α antagonist is administered intranasally to the mammal.

In a further embodiment, the TNF-α antagonist is administered systemically. The TNF-α antagonist can be administered, for example, intravenously, parenterally, subcutaneously, intramuscularly, ophthalmicly, intraventricularly, intraperitoneally, orally, or topically, to said mammal. In a further aspect, the TNF-α antagonist is administered in an encapsulated form in a lipophilic compound or liposome. In a further aspect, the TNF-α antagonist is encapsulated in a polymer.

### BRIEF DESCRIPTION OF THE DRAWINGS

FIGS. 1A, 1B, and 1C show phosphorylation of p38 MAP kinase in the spinal cord.

FIG. 2 shows a Western blot of rat spinal cord lysates.

FIG. 3 shows the effect of intrathecal p38 inhibitor on paw swelling in adjuvant arthritis.

FIG. 4 shows the effect of intrathecal p38 inhibitor on histologic joint damage in adjuvant arthritis.

FIG. 5 shows the effect of intrathecal p38 inhibitor on radiographic joint damage in adjuvant arthritis.

FIG. 6 shows the effect of intrathecal p38 inhibitor on joint gene expression.

FIG. 7 shows the effect of intrathecal etanercept on adjuvant arthritis.

### DETAILED DESCRIPTION

The methods of the present invention for preventing or treating inflammatory disease in a mammal are based on the finding of a intracellular mechanism implicating central nervous system (CNS)-derived MAP kinases, for example, p38 MAP kinase, in the regulation of peripheral inflammation. Results indicate that selective CNS blockade of p38 MAPK or selective CNS blockade of tumor necrosis factor α (TNF-α) has profound effects on peripheral inflammation, arthritis, and joint destruction.

The present invention provides a method for preventing or treating inflammatory disease or arthritis in a mammal by administering an inhibitor of a signal transduction enzyme wherein the inhibitor is targeted to the central nervous system (CNS) of the mammal. The signal transduction enzyme within the CNS can be a mitogen activated protein (MAP) kinase, for example, a p38 MAP kinase. MAP kinases within the CNS provide a connection between the CNS and peripheral inflammation that can have major implications for therapy for prevention or treatment of inflammatory disease in the mammal. The present invention further provides a method for preventing or treating inflammatory disease or arthritis in a mammal by administering a TNF-α antagonist wherein the TNF-α antagonist is targeted to the central nervous system (CNS) of the mammal. By targeting therapy to the CNS, the potential for toxicity of MAP kinase inhibitors or TNF-α antagonists, related to suppressed host defense or interference with key signaling events in other major organs, can be decreased. The methods provide delivery of therapeutic agents delivered directly into the CNS or specifically designed for preferential distribution into the CNS. The MAP kinase inhibitor or TNF-α antagonist is targeted to the CNS by methods of administration including, but not limited to, direct injection into the CNS, intranasal administration to the CNS, or parenteral administration of pharmaceutical compositions targeted via receptors or antibodies to the CNS. Development of MAP kinase inhibitors or TNF-α antagonists for treatment of inflammatory disease or arthritis has been hampered by toxicity to the liver and other organs. The methods of the present invention minimize toxic side effects of MAP kinase inhibitors or TNF-α antagonists for treatment of disease.

Based on studies demonstrating that nerve injury is associated with activation of MAP kinases and cytokine expression in the CNS and that p38 blockade has analgesic activities in animal models, (Kim, et al., Neuroreport., 13: 2483-2486, 2002), studies were performed to asked whether there is a corresponding effect on peripheral inflammation. The carrageenan rat paw edema model of inflammation (CPE) was used because this stimulus appears to enhance p38 activation in the CNS. Surprisingly, administration of a p38 inhibitor into the intrathecal space decreased paw swelling even though systemic administration of the same dose had no effect. This is the first suggestion that spinal MAPKs, or p38 in particular, can influence peripheral inflammatory responses in vivo. The lineage of cells that express p38 and the neural networks that are responsible for this action are unknown and represent a significant focus of the current proposal.

Subsequently, the activation state of p38 in the spinal cord was examined during the evolution of chronic inflammation. The adjuvant arthritis model causes severe destructive polyarticular arthritis and is often used to evaluate therapeutic agents for rheumatoid arthritis (RA). Initial studies showed that p38 is expressed in the spinal cord and DRGs, and is phosphorylated about 1 week after immunization in specific locations of the spinal cord dorsal horn. Both neurons and microglial cells in the spinal cord contain P-p38, although double staining studies suggest that the latter is the primary location. A therapeutic intervention with intrathecal SB203580, a commonly-used water soluble p38 inhibitor that is amenable to use in this system, markedly decreased paw swelling. More striking, inhibition of p38 in the CNS also decreased histologic evidence of synovial inflammation and suppressed radiographic signs of bone and cartilage destruction. In addition to determining the central mechanisms responsible for this striking observation, the influence of p38 activation in the spinal cord on pathogenic processes in the synovium will be studied.

Systemic p38 blockade has been extensively studied in inflammation and clearly demonstrates benefit in several models of arthritis. Nishikawa, et al., Arthritis Rheum., 48: 2670-2681, 2003; Jackson, et al., J. Pharmacol. Exp. Ther., 284: 687-692, 1998; Badger, et al., J. Pharmacol. Exp. Ther., 279: 1453-1461, 1996. Of interest, the biological effects are similar to those observed in studies evaluating IT administration of SB203580. Not only is synovial inflammation decreased, but also joint damage is markedly attenuated. What has been especially striking is that very high doses of p38 inhibitors are required when delivered by oral or parenteral routes. Typically, 30 to 100 mg/kg are needed even though the compounds have excellent pharmacokinetics and inhibit the enzyme at nanomolar concentrations. The effective dose in vivo is several hundred-fold higher than the amount required via IT therapy. These observations raise the intriguing possibility that the CNS might be, in part, responsible for the anti-inflammatory effects seen with systemic administration of p38 inhibitors, and that inadequate CNS penetration accounts for this discrepancy.

The present invention provides a connection between the CNS and peripheral inflammation that can have major implications for therapy for prevention or treatment of inflammatory disease in a mammal. In one aspect of the invention, inflammation in the peripheral tissues is sensed by the CNS, most likely through somatic afferent pathways, resulting in activation of spinal cord MAPKs (for example, p38). This intracellular signaling pathway can subsequently relay information to the periphery, which is essential for full expression of somatic host responses. By analogy to nociception, it is also possible that local TNF-α production might either result from spinal p38 activation or that TNF-α further activates p38 in the CNS, thereby modulating peripheral inflammation. These pathways could be related (either in series or in parallel), thereby providing a unified mechanism that permits the CNS to sample the periphery and through homeostatic neural reflexes modify host responses. It is possible, however, that p38 is a key link between NMDA and the anti-inflammatory efferent pathway to the periphery. If so, then these studies will answer key questions on the mechanism of glutamate receptor regulation of inflammation.

In addition to providing insights on the interactions between the CNS and host defense, this MAPK-dependent pathway can significantly influence the design of MAPK inhibitors. The actions of p38 inhibitors, which are currently in early clinical development, may depend on CNS penetration for full anti-inflammatory and analgesic effects. Understanding the mechanisms of the anti-inflammatory actions can also lead to the identification of additional pathways that will have therapeutic utility (e.g., other MAP kinases). Although these studies do evaluate effects on cognitive function, the ability of neural reflexes to alter peripheral inflammation and tissue destruction can potentially contribute to variability in clinical responses to therapeutic agents. For instance, the well documented “placebo” responses and the waxing and waning disease course in diseases like rheumatoid arthritis could relate to counter-regulatory mechanisms that are superimposed on traditional pharmacologic approaches. The influence of MAP kinases in the CNS on the natural history of inflammation will be characterized, and the molecular mechanisms that contribute to the neural control over distant processes will be dissected.

“Inhibitors,” “activators,” and “modulators” of MAP kinase signaling refer to inhibitory, activating, or modulating molecules, respectively, identified using in vitro and in vivo assays for MAP kinase binding or signaling, e.g., ligands, agonists, antagonists, and their homologs and mimetics.

“Modulator” includes inhibitors and activators. Inhibitors refers to agents that, e.g., bind to, partially or totally block stimulation, decrease, prevent, delay activation, inactivate, desensitize, or down regulate the activity of MAP kinase signaling, e.g., antagonists. Activators are agents that, e.g., bind to, stimulate, increase, open, activate, facilitate, enhance activation, sensitize or up regulate the activity of MAP kinase signaling, e.g., agonists. Modulators include agents that, e.g., alter the interaction of MAP kinase with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring MAP kinase ligands, e.g., with altered activity, as well as naturally occurring and synthetic ligands, antagonists, agonists, small chemical molecules and the like. Such assays for inhibitors and activators include, e.g., applying putative modulator compounds to a cell expressing a MAP kinase and then determining the functional effects on MAP kinase signaling, as described herein. Samples or assays comprising MAP kinase that are treated with a potential activator, inhibitor, or modulator are compared to control samples without the inhibitor, activator, or modulator to examine the extent of inhibition. Control samples (untreated with inhibitors) can be assigned a relative MAP kinase activity value of 100%. Inhibition of MAP kinase is achieved when the MAP kinase activity value relative to the control is about 80%, optionally 50% or 25-0%. Activation of MAP kinase is achieved when the MAP kinase activity value relative to the control is 110%, optionally 150%, optionally 200-500%, or 1000-3000% higher. Exemplary MAP kinase binding activity assays of the present invention are: a MAP kinase ligand blot assay (Aymerich et al., Invest Opthalmol Vis Sci. 42:3287-93, 2001); a MAP kinase affinity column chromatography assay (Alberdi et al., J Biol Chem. 274:31605-12, 1999) and a MAP kinase ligand binding assay (Alberdi et al., J Biol Chem. 274:31605-12, 1999). Each incorporated by reference in their entirety.

“Signaling in cells” refers to the interaction of a ligand, such as an endogenous or exogenous ligand, e.g., diacylglycerides including lipotechoic acid (LTA) and S-MALP-2, with MAP kinase resulting in cell signaling to produce a response, for example, an inflammatory response.

“Test compound” refers to a nucleic acid, DNA, RNA, protein, polypeptide, or small chemical entity that is determined to effect an increase or decrease in a gene expression as a result of signaling through the MAP kinase pathway. The test compound can be an antisense RNA, ribozyme, polypeptide, or small molecular chemical entity. The term “test compound” can be any small chemical compound, or a biological entity, such as a protein, sugar, nucleic acid or lipid. Typically, test compounds will be small chemical molecules and polypeptides. A “test compound specific for signaling by MAP kinase is determined to be a modulator of MAP kinase pathway signaling via ligand.

“Cell-based assays” include MAP kinase binding assays, for example, radioligand or fluorescent ligand binding assays for MAP kinase to cells, plasma membranes, detergent-solubilized plasma membrane proteins, immobilized collagen (Alberdi et al., 1999, JBC; Meyer et al., 2002); MAP kinase-affinity column chromatography (Alberdi et al., 1999, JBC; Aymerich et al., 2001); MAP kinase ligand blot using a radio- or fluoresceinated-ligand (Aymerich et al., 2001; Meyer et al., 2002); Size-exclusion ultrafiltration (Alberdi et al., 1998, Biochem.; Meyer et al., 2002); or ELISA. Cell-based assays further include, but are not limited to TNF cellular assay, MAP kinase binding assay, fatty acid translocator assay, or thrombospondin binding assay.

“Detecting an effect” refers to an effect measured in a cell-based assay system. For example, the effect detected can be MAP kinase activity in an assay system, for example, MAP kinase binding assay.

“Assay being indicative of modulation” refers to results of a cell-based assay system indicating that inhibition of cell activation by MAP kinase pathway signaling induces a protective response in cells against inflammation.

“Biological activity” and “biologically active” with regard to a MAP kinase activity of the present invention refer to the ability of a molecule to specifically bind to and signal through a native or recombinant MAP kinase, or to block the ability of a native or recombinant MAP kinase to participate in signal transduction. Thus, the (native and variant) MAP kinase ligands of the present invention include agonists and antagonists of a native or recombinant MAP kinase. Preferred biological activities of the MAP kinase ligands of the present invention include the ability to inhibit, for example, an inflammatory disease or an inflammatory response. Accordingly, the administration of the compounds or agents of the present invention can prevent or delay, to alleviate, or to arrest or inhibit development of the symptoms or conditions associated with atherosclerosis, autoimmune disease, Alzheimer's disease, or other disorders.

“High affinity” for a ligand refers to an equilibrium association constant (Ka) of at least about 10³M⁻¹, at least about 10⁴M⁻¹, at least about 10⁵M⁻¹, at least about 10⁶M⁻¹, at least about 10⁷M⁻¹, at least about 10⁸M⁻¹, at least about 10⁹M⁻¹, at least about 10¹⁰M⁻¹, at least about 10¹¹M⁻¹, or at least about 10¹²M⁻¹ or greater, e.g., up to 10¹³M⁻¹ or 1014M⁻¹ or greater. However, “high affinity” binding can vary for other ligands.

“Kₐ”, as used herein, is intended to refer to the equilibrium association constant of a particular ligand-receptor interaction, e.g., antibody-antigen interaction. This constant has units of 1/M.

“K_{d}”, as used herein, is intended to refer to the equilibrium dissociation constant of a particular ligand-receptor interaction. This constant has units of M.

“kₐ”, as used herein, is intended to refer to the kinetic association constant of a particular ligand-receptor interaction. This constant has units of 1/Ms.

“k_{d}”, as used herein, is intended to refer to the kinetic dissociation constant of a particular ligand-receptor interaction. This constant has units of 1/s.

“Particular ligand-receptor interactions” refers to the experimental conditions under which the equilibrium and kinetic constants are measured.

“Isotype” refers to the antibody class that is encoded by heavy chain constant region genes. Heavy chains are classified as gamma, mu, alpha, delta, or epsilon, and define the antibody's isotype as IgG, IgM, IgA, IgD and IgE, respectively. Additional structural variations characterize distinct subtypes of IgG (e.g., IgG₁, IgG₂, IgG₃ and IgG₄) and IgA (e.g., IgA₁ and IgA₂)

“Antagonist” is used in the broadest sense, and includes any molecule that partially or fully blocks, inhibits, or neutralizes a biological activity of a MAP kinase polypeptide. In a similar manner, the term “agonist” is used in the broadest sense and includes any molecule that mimics or enhances a biological activity of a MAP kinase polypeptide. Suitable antagonist molecules specifically include antagonist antibodies or antibody fragments, fragments or amino acid sequence variants of native MAP kinase polypeptides, peptides, antisense oligonucleotides, small organic molecules, and the like. Methods for identifying antagonists of a MAP kinase polypeptide can comprise contacting a MAP kinase polypeptide with a candidate antagonist molecule and measuring a detectable change in one or more biological activities normally associated with the MAP kinase polypeptide.

The ability of a molecule to bind to MAP kinase can be determined, for example, by the ability of the putative ligand to bind to MAP kinase immunoadhesin coated on an assay plate. Specificity of binding can be determined by comparing binding to MAP kinase.

“Sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

“Cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny cannot be precisely identical in DNA content, due to deliberate or inadvertent mutations. Mutant progeny that have the same function or biological activity as screened for in the originally transformed cell are included. Where distinct designations are intended, it will be clear from the context.

“Treating” refers to any indicia of success in the treatment or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination. Accordingly, the term “treating” includes the administration of the compounds or agents of the present invention to inhibit inflammatory disease or autoimmune disease. It also includes the administration of the compounds of the present invention to promote inflammation in a subject toward infection with a pathogen. Accordingly, the term “treating” includes the administration of the compounds or agents of the present invention to prevent or delay, to alleviate, or to arrest or inhibit development of the symptoms or conditions associated with atherosclerosis, autoimmune disease, Alzheimer's disease, or other disorders. The term “therapeutic effect” refers to the reduction, elimination, or prevention of the disease, symptoms of the disease, or side effects of the disease in the subject.

“Periphery of a mammal” refers to any part of the body outside the central nervous system of the mammal.

“Concomitant administration” of a known drug with a compound of the present invention means administration of the drug and the compound at such time that both the known drug and the compound will have a therapeutic effect or diagnostic effect. Such concomitant administration can involve concurrent (i.e., at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

“Inflammation” or “inflammatory response” refers to an innate immune response that occurs when tissues are injured by bacteria, trauma, toxins, heat, or any other cause. The damaged tissue releases compounds including histamine, bradykinin, and serotonin. Inflammation refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

“Lipid” refers to a fat or fat-like substance that is insoluble in polar solvents such as water. The term “lipid” is intended to include true fats (e.g., esters of fatty acids and glycerol); lipids (phospholipids, cerebrosides, waxes); sterols (cholesterol, ergosterol) and lipoproteins (e.g., HDL, LDL and VLDL). “Lipid” can also refer to a synthetic or naturally-occurring amphipathic compound which comprises a hydrophilic component and a hydrophobic component. Lipids include, for example, fatty acids, neutral fats, phosphatides, glycolipids, aliphatic alcohols and waxes, terpenes and steroids.

“Subject”, “patient” or “mammal” refer to any mammalian patient or subject to which the compositions of the invention can be administered. The term mammal refers to human patients and non-human primates, as well as experimental animals such as rabbits, rats, and mice, and other animals. In an exemplary embodiment, of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that can be associated with the targeted or suspected disease or condition. These and other routine methods allow the clinician to select patients in need of therapy using the methods and formulations of the invention.

By “solid phase” is meant a non-aqueous matrix to which a reagent of interest (e.g., MAP kinase or an antibody thereto) can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

“Specifically (or selectively) binds” to an antibody refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

“Specifically bind(s)” or “bind(s) specifically” when referring to a peptide or small molecule refers to a peptide molecule or small molecule which has intermediate or high binding affinity, exclusively or predominately, to a target molecule. The phrase “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions can require a binding moiety that is selected for its specificity for a particular target antigen. A variety of assay formats can be used to select ligands that are specifically reactive with a particular protein. For example, solid-phase ELISA immunoassays, immunoprecipitation, Biacore and Western blot are used to identify peptides that specifically react with MAP kinase domain-containing proteins. Typically a specific or selective reaction will be at least twice background signal or noise and more typically more than 10 times background. Specific binding between a monovalent peptide and MAP kinase means a binding affinity of at least 10³ M⁻¹, and preferably 10⁵, 10⁶, 10⁷, 10⁸, 10⁹ or 10¹⁰ M⁻¹.

This invention relies on routine techniques in the field of recombinant genetics. Basic texts disclosing the general methods of use in this invention include Sambrook et al., Molecular Cloning, A Laboratory Manual, 2nd ed., 1989; Kriegler, Gene Transfer and Expression: A Laboratory Manual, 1990; and Ausubel et al., eds., Current Protocols in Molecular Biology, 1994.

MAP kinase nucleic acids, polymorphic variants, orthologs, and alleles that are substantially identical to sequences provided herein can be isolated using MAP kinase nucleic acid probes and oligonucleotides under stringent hybridization conditions, by screening libraries. Alternatively, expression libraries can be used to clone MAP kinase protein, polymorphic variants, orthologs, and alleles by detecting expressed homologs immunologically with antisera or purified antibodies made against human MAP kinase or portions thereof.

### Inhibitors of p38 MAP Kinase

“Inhibitor” includes, but is not limited to, any suitable molecule, compound, protein or fragment thereof, nucleic acid, formulation or substance that can regulate p38 MAP kinase activity. The inhibitor can affect a single p38 MAP kinase isoform (p38α, p38β, p38γ, and p38.delta.), more than one isoform, or all isoforms of p38 MAP kinase. In a preferred embodiment, the inhibitor regulates the isoform of p38 MAP kinase.

According to the present invention, it is contemplated that the inhibitor can exhibit its regulatory effect upstream or downstream of p38 MAP kinase or on p38 MAP kinase directly. Examples of inhibitor regulated p38 MAP kinase activity include those where the inhibitor can decrease transcription and/or translation of p38 MAP kinase, can decrease or inhibit post-translational modification and/or cellular trafficking of p38 MAP kinase, or can shorten the half-life of p38 MAP kinase. The inhibitor can also reversibly or irreversibly bind p38 MAP kinase, inactivate its enzymatic activity, or otherwise interfere with its interaction with downstream substrates.

If acting on p38 MAP kinase directly, in one embodiment the inhibitor should exhibit an IC₅₀ value of about 5 μM or less, preferably 500 nm or less, more preferably 100 nm or less. In a related embodiment, the inhibitor should exhibit an IC₅₀ value relative to the p38α. MAP kinase isoform that is about ten fold less than that observed when the same inhibitor is tested against other p38 MAP kinase isoforms in a comparable assay.

Those skilled in the art can determine whether or not a compound is useful in the present invention by evaluating its p38 MAP kinase activity as well as its relative IC₅₀ value. This evaluation can be accomplished through conventional in vitro assays. In vitro assays include assays that assess inhibition of kinase or ATPase activity of activated p38 MAP kinase. In vitro assays can also assess the ability of the inhibitor to bind p38, MAP kinase or to reduce or block an identified downstream effect of activated p38 MAP kinase, e.g., cytokine secretion. IC₅₀ values are calculated using the concentration of inhibitor that causes a 50% decrease as compared to a control.

A binding assay is a fairly inexpensive and simple in vitro assay to run. As previously mentioned, binding of a molecule to p38 MAP kinase, in and of itself, can be inhibitory, due to steric, allosteric or charge-charge interactions. A binding assay can be performed in solution or on a solid phase using p38 MAP kinase or a fragment thereof as a target. By using this as an initial screen, one can evaluate libraries of compounds for potential p38 MAP kinase regulatory activity.

The target in a binding assay can be either free in solution, fixed to a support, or expressed in or on the surface of a cell. A label (e.g., radioactive, fluorescent, quenching) can be placed on the target, compound, or both to determine the presence or absence of binding. This approach can also be used to conduct a competitive binding assay to assess the inhibition of binding of a target to a natural or artificial substrate or binding partner. In any case, one can measure, either directly or indirectly, the amount of free label versus bound label to determine binding. There are many known variations and adaptations of this approach to minimize interference with binding activity and optimize signal.

For purposes of in vitro cellular assays, the compounds that represent potential inhibitors of p38 MAP kinase function can be administered to a cell in any number of ways. Preferably, the compound or composition can be added to the medium in which the cell is growing, such as tissue culture medium for cells grown in culture. The compound is provided in standard serial dilutions or in an amount determined by analogy to known modulators. Alternatively, the potential inhibitor can be encoded by a nucleic acid that is introduced into the cell wherein the cell produces the potential inhibitor itself.

Alternative assays involving in vitro analysis of potential inhibitors include those where cells (e.g., HeLa) transfected with DNA coding for relevant kinases can be activated with substances such as sorbitol, IL-1, TNF, or PMA. After immunoprecipitation of cell lysates, equal aliquots of immune complexes of the kinases are pre-incubated for an adequate time with a specific concentration of the potential inhibitor followed by addition of kinase substrate buffer mix containing labeled ATP and GST-ATF2 or MBP. After incubation, kinase reactions are terminated by the addition of SDS loading buffer. Phosphorylated substrate is resolved through SDS-PAGE and visualized and quantitated in a phosphorimager. The p38 MAP kinase regulation, in terms of phosphorylation and IC₅₀ values, can be determined by quantitation. See e.g., Kumar, S. et al., Biochem. Biophys. Res. Commun. 235:533-538 (1997).

Other in vitro assays can also assess the production of TNF-α as a correlation to p38 MAP kinase activity. One such example is a Human Whole Blood Assay. In this assay, venous blood is collected from, e.g., healthy male volunteers into a heparinized syringe and is used within 2 hours of collection. Test compounds are dissolved in 100% DMSO and 1 μl aliquots of drug concentrations ranging from 0 to 1 mM are dispensed into quadruplicate wells of a 24-well microtiter plate (Nunclon Delta SI, Applied Scientific Co., San Francisco, Calif.). Whole blood is added at a volume of 1 ml/well and the mixture is incubated for 15 minutes with constant shaking (Titer Plate Shaker, Lab-Line Instruments, Inc., Melrose Park, Ill.) at a humidified atmosphere of 5% CO₂ at 37° C. Whole blood is cultured either undiluted or at a final dilution of 1:10 with RPMI 1640 (Gibco 31800+NaHCO₃, Life Technologies, Rockville, Md. and Scios, Inc., Sunnyvale, Calif.). At the end of the incubation period, 10 μl of LPS (E. coli 0111:B4, Sigma Chemical Co., St. Louis, Mo.) is added to each well to a final concentration of 1 or 0.1 μg/ml for undiluted or 1:10 diluted whole blood, respectively. The incubation is continued for an additional 2 hours. The reaction is stopped by placing the microtiter plates in an ice bath, and plasma or cell-free supernates are collected by centrifugation at 3000 rpm for 10 minutes at 4° C. The plasma samples are stored at −80° C. until assayed for TNF-α levels by ELISA, following the directions supplied by Quantikine Human TNF-α assay kit (R&D Systems, Minneapolis, Minn.). IC₅₀ values are calculated using the concentration of inhibitor that causes a 50% decrease as compared to a control.

A similar assay is an enriched mononuclear cell assay. The enriched mononuclear cell assay begins with cryopreserved Human Peripheral Blood Mononuclear Cells (HPBMCs) (Clonetics Corp.) that are rinsed and resuspended in a warm mixture of cell growth media. The resuspended cells are then counted and seeded at 1.times. 10⁶ cells/well in a 24-well microtitre plate. The plates are then placed in an incubator for an hour to allow the cells to settle in each well. After the cells have settled, the media is aspirated and new media containing 100 ng/ml of the cytokine stimulatory factor Lipopolysaccharide (LPS) and a test chemical compound is added to each well of the microtiter plate. Thus, each well contains HPBMCs, LPS and a test chemical compound. The cells are then incubated for 2 hours, and the amount of the cytokine Tumor Necrosis Factor α (TNF-α) is measured using an Enzyme Linked Immunoassay (ELISA). One such ELISA for detecting the levels of TNF-α is commercially available from R&D Systems. The amount of TNF-α production by the HPBMCs in each well is then compared to a control well to determine whether the chemical compound acts as an inhibitor of cytokine production.

Exemplary inhibitors of p38 MAP kinase include but are not limited to, small molecule inhibitors, antisense oligonucleotide inhibitors, antibody inhibitors, protein inhibitors. See, for example, U.S. Pat. Nos. 6,696,471, U.S. Pat. No. 6,696,443, U.S. Pat. No. 6,630,485, U.S. Pat. No. 6,617,324, U.S. Pat. No. 6,579,873, U.S. Pat. No. 6,525,059, U.S. Pat. No. 6,509,361, U.S. Pat. No. 6,509,361, U.S. Pat. No. 6,479,507, U.S. 6,448,079, U.S. Pat. No. 6,444,696, each incorporated herein by reference in their entirety.

### Targeting MAP Kinase Inhibitors or TNF-α Antagonists to the Central Nervous System

The present invention provides methods for the efficient delivery of a therapeutic amount of a MAP kinase inhibitor or TNF-α antagonist to the central nervous system of a mammal. A number of delivery methods are useful in the present invention. The pharmaceutical composition comprising the MAP kinase inhibitor or TNF-α antagonist can be delivered via the ocular route (U.S. patent application 20030181354, incorporated herein by reference in its entirety), the intranasal route (U.S. patent application 20030225031, incorporated herein by reference in its entirety), or the pharmaceutical composition can be delivered systemically while being targeted to the CNS. For example, viral vectors, such as AAV, can transport a transgene to the central nervous system of the mammal. Viral vector delivery can be enhanced by convection-enhanced delivery devices, for example, osmotic or infusion pumps. U.S. patent application 20020141980, incorporated herein by reference in its entirety. Viral vectors can be delivered to many cells over large areas of the brain. Moreover, the delivered vectors efficiently express transgenes in CNS cells (e.g., neurons or glial cells).

Antibodies in combination with liposomes can be used to target therapeutic MAP kinase inhibitors or TNF-α antagonists across the blood brain barrier into the CNS. Antibodies that bind to cell surface receptors of cells of the blood brain barrier are useful for transporting therapeutic MAP kinase inhibitors or TNF-α antagonists across the blood brain barrier into the CNS Small antibody fragments have essential characteristics of brain-specific delivery vectors and can be used to facilitate drug transport across the BBB. U.S. patent application 20040161738, incorporated herein by reference in its entirety.

### Identification of Compounds for Treatment and Prophylaxis of Disease

Identifying bioactive agents that modulate MAP kinase activity, the information is used in a wide variety of ways. In one method, one of several cellular assays, e.g., MAP kinase activity assay, can be used in conjunction with high throughput screening techniques, to allow monitoring for antagonists of MAP kinase pathway signaling after treatment with a candidate agent, Zlokarnik, et al., Science 279:84-8, 1998; and Heid et al., Genome Res. 6:986, 1996; each incorporated herein by reference in their entirety. In one method, the candidate agents are added to cells.

“Candidate bioactive agent” or “drug candidate” or grammatical equivalents as used herein describes any molecule, e.g., protein, oligopeptide, small organic molecule, polysaccharide, polynucleotide, to be tested for bioactive agents that are capable of directly or indirectly altering the activity of MAP kinase pathway signaling. In one method, the bioactive agents modulate MAP kinase pathway signaling. In a further embodiment of the method, the candidate agents induce an antagonist effect in a MAP kinase activity assay, as further described below. Generally a plurality of assay mixtures are run in parallel with different agent concentrations to obtain a differential response to the various concentrations. Typically, one of these concentrations serves as a negative control, i.e., at zero concentration or below the level of detection.

Candidate agents encompass numerous chemical classes, though typically they are organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents can be subjected to directed or random chemical modifications, such as acylation, alkylation, esterification, amidification to produce structural analogs.

In some embodiments, the candidate bioactive agents are proteins. By “protein” herein is meant at least two covalently attached amino acids, which includes proteins, polypeptides, oligopeptides and peptides. The protein can be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the methods herein. “Amino acid” also includes imino acid residues such as proline and hydroxyproline. The side chains can be in either the (R) or the (S) configuration. In further embodiments, the amino acids are in the (S) or (L) configuration. If non-naturally occurring side chains are used, non-amino acid substituents can be used, for example to prevent or retard in vivo degradations.

In one method, the candidate bioactive agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and mammalian proteins, and human proteins.

In some methods, the candidate bioactive agents are peptides of from about 5 to about 30 amino acids, typically from about 5 to about 20 amino acids, and typically from about 7 to about 15 being. The peptides can be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they can incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In some methods, the library can be fully randomized, with no sequence preferences or constants at any position. In other methods, the library can be biased. Some positions within the sequence are either held constant, or are selected from a limited number of possibilities. For example, in some methods, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, or to purines. In other methods, the candidate bioactive agents are nucleic acids, as defined above.

As described above generally for proteins, nucleic acid candidate bioactive agents can be naturally occurring nucleic acids, random nucleic acids, or “biased” random nucleic acids. For example, digests of procaryotic or eucaryotic genomes can be used as is outlined above for proteins. In some methods, the candidate bioactive agents are organic chemical moieties.

Several different drug screening methods can be accomplished to identify drugs or bioactive agents that modulate MAP kinase activity. One such method is the screening of candidate agents that can act as an antagonist of MAP kinase activity, thus generating the associated phenotype. Candidate agents that can act as an antagonist to MAP kinase pathway signaling, as shown herein, is expected to result in the anti-inflammatory phenotype. Thus, in some methods, candidate agents can be determined that mimic or alter MAP kinase pathway signaling.

In other methods, screening can be done to alter the biological function of the MAP kinase signaling. Having identified the importance of a MAP kinase signaling, screening for agents that bind and/or modulate the biological activity of the MAP kinase can be performed as outlined below.

Thus, screening of candidate agents that modulate MAP kinase pathway signaling either at the level of gene expression or protein level can be accomplished.

In some methods, a candidate agent can be administered in any one of several cellular assays, e.g., MAP kinase activity assay. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e., a peptide) can be put into a viral construct such as a retroviral construct and added to the cell, such that expression of the peptide agent is accomplished; see PCT US97/01019, incorporated herein by reference in its entirety.

### Liposome Pharmaceutical Compositions

“Liposome” or “lipophilic compound” refer to unilamellar vesicles or multilamellar vesicles such as are described in U.S. Pat. No. 4,753,788 and U.S. Application No. 2004/0156889. “Unilamellar liposomes,” also referred to as “single lamellar vesicles,” are spherical vesicles that includes one lipid bilayer membrane which defines a single closed aqueous compartment. The bilayer membrane includes two layers of lipids; an inner layer and an outer layer (leaflet). The outer layer of the lipid molecules are oriented with their hydrophilic head portions toward the external aqueous environment and their hydrophobic tails pointed downward toward the interior of the liposome. The inner layer of the lipid lays directly beneath the outer layer, the lipids are oriented with their heads facing the aqueous interior of the liposome and their tails toward the tails of the outer layer of lipid.

“Multilamellar liposomes,” also referred to as “multilamellar vesicles” or “multiple lamellar vesicles,” include more than one lipid bilayer membrane, which membranes define more than one closed aqueous compartment. The membranes are concentrically arranged so that the different membranes are separated by aqueous compartments, much like an onion.

“Encapsulation” and “entrapped” refer to the incorporation or association of the pharmaceutical agent in or with a liposome. The pharmaceutical agent may be associated with the lipid bilayer or present in the aqueous interior of the liposome, or both. In one embodiment, a portion of the encapsulated pharmaceutical agent takes the form of a precipitated salt in the interior of the liposome. The pharmaceutical agent may also self precipitate in the interior of the liposome.

“Excipient” “counterion” and “counterion excipient,” refer to a substance that can initiate or facilitate drug loading and may also initiate or facilitate precipitation of the pharmaceutical agent in the aqueous interior of the liposome. Examples of excipients include, but are not limited to, the acid, sodium or ammonium forms of monovalent anions such as chloride, acetate, lactobionate and formate; divalent anions such as aspartate, succinate and sulfate; and trivalent ions such as citrate and phosphate. Preferred excipients include citrate and sulfate.

“Phospholipid” refers to any one phospholipid or combination of phospholipids capable of forming liposomes. Phosphatidylcholines (PC), including those obtained from egg, soy beans or other plant sources or those that are partially or wholly synthetic, or of variable lipid chain length and unsaturation are suitable for use in the present invention. Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in this invention. All of these phospholipids are commercially available. Preferred PCs are HSPC and DSPC; the most preferred is HSPC.

Further, phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in formulations. Other suitable phospholipids include phosphatidylethanolamines phosphatidylinositols, and phosphatidic acids containing lauric, myristic, stearoyl, and palmitic acid chains. Further, incorporation of polyethylene glycol (PEG) containing phospholipids is also contemplated by the present invention.

“Parenteral” refers to intravenous (IV), intramuscular (IM), subcutaneous (SubQ) or intraperitoneal (IP) administration.

“Improved therapeutic index” refers to a higher therapeutic index relative to the free drug. The therapeutic index can be expressed as a ratio of the lethal dose for 50% of the animals relative to the effective dose.

“Treat” or “treating” refers to: (i) preventing a pathologic condition (e.g., inflammatory disease) from occurring (e.g. prophylaxis) or symptoms related to the same; (ii) inhibiting the pathologic condition or arresting its development or symptoms related to the same; or (iii) relieving the pathologic condition or symptoms related to the same.

It is contemplated by this invention to optionally include cholesterol in the liposomal formulation. Cholesterol is known to improve liposome stability and prevent loss of phospholipid to lipoproteins in vivo.

Any suitable lipid: pharmaceutical agent ratio that is efficacious is contemplated by this invention. Preferred lipid: pharmaceutical agent molar ratios include about 5:1 to about 100:1, more preferably about 10:1 to about 40:1. The most preferred lipid: pharmaceutical agent molar ratios include about 15:1 to about 25:1. Preferred liposomal formulations include phospholipid:cholesterol molar ratios over the range of 1.5:0.5 to 2:1.5. Most preferred liposomal formulation is 2:1 PC:chol with or without 1 to 4 mole percent of a phosphatidylglycerol. The most preferred liposomal size is less than 100 nm. The preferred loading efficiency of pharmaceutical agent is a percent encapsulated pharmaceutical agent of about 70% or greater. Encapsulation includes molecules present in the interior aqueous space of the liposome, molecules in the inner or outer leaflet of the membrane bilayer, molecules partially buried in the outer leaflet of the bilayer and partially external to the liposome, and molecules associated with the surface of the liposome, e.g., by electrostatic interactions.

Generally, the process of preparing the formulation embodied in the present invention is initiated with the preparation of a solution from which the liposomes are formed. This is done, for example, by weighing out a quantity of a phosphatidylcholine optionally cholesterol and optionally a phosphatidylglycerol and dissolving them in an organic solvent, preferably chloroform and methanol in a 1:1 mixture (v/v) or alternatively neat chloroform. The solution is evaporated to form a solid lipid phase such as a film or a powder, for example, with a rotary evaporator, spray dryer or other means. The film or powder is then hydrated with an aqueous solution containing an excipient having a pH range from 2.0 to 7.4 to form a liposome dispersion. The preferred aqueous solution for purposes of hydration is a buffered solution of the acid, sodium or ammonium forms of citrate or sulfate. The preferred buffers are up to about 60 mM, citric acid (pH 2.0-5.0), ammonium citrate (pH 2.0-5.5), or ammonium sulfate (pH 2.0 to 5.5). It would be known by one of skill in the art that other anionic acid buffers could be used, such as phosphoric acid. The lipid film or powder dispersed in buffer is heated to a temperature from about 25° C. to about 70° C. depending on the phospholipids used.

The liposomes formed by the procedure of the present invention can be lyophilized or dehydrated in the presence of a hydrophilic agent.

Multilamellar liposomes are formed by agitation of the dispersion, preferably through the use of a thin-film evaporator apparatus such as is described in U.S. Pat. No. 4,935,171 or through shaking or vortex mixing. Unilamellar vesicles are formed by the application of a shearing force to an aqueous dispersion of the lipid solid phase, e.g., by sonication or the use of a microfluidizing apparatus such as a homogenizer or a French press. Shearing force can also be applied using either injection, freezing and thawing, dialyzing away a detergent solution from lipids, or other known methods used to prepare liposomes. The size of the liposomes can be controlled using a variety of known techniques including the duration of shearing force. Preferably, a homogenizing apparatus is employed to from unilamellar vesicles having diameters of less than 200 nanometers at a pressure of 3,000 to 14,000 psi preferably 10,000 to 14,000 psi, and a temperature of about the aggregate transition temperature of the lipids.

Unentrapped excipient may or may not be removed or exchanged from the liposome dispersion by buffer exchange to 9% sucrose using either dialysis, size exclusion column chromatography (Sephadex G-50 resin) or ultrafiltration (100,000-300,000 molecular weight cut off). Each preparation of small unilamellar liposomes is then actively loaded with drug, for approximately 10-30 minutes against a gradient, such as a membrane potential, generated as the external pH is titrated to the range of 5.0 or above with sodium hydroxide. The temperature ranges during the drug loading step is generally between about 50° C.-70° C. with lipid:drug ratios between 5:1 and 100:1. Unentrapped pharmaceutical agent is removed from the liposome dispersion by buffer exchange to 9% sucrose using either dialysis, size exclusion column chromatography (Sephadex G-50 resin) or ultrafiltration (100,000-300,000 molecular weight cut off). Samples are generally filtered at about 55° C.-65° C. through a 0.22 micron filter composed of either cellulose acetate or polyether sulfone.

As described above, the pharmaceutical agent is generally loaded into pre-formed liposomes using known loading procedures (see for example, Deamer et al., BBA 274:323-335 (1972); Forssen, U.S. Pat. No. 4,946,683; Cramer et al., BBRC 75:295-301 (1977); Bally, U.S. Pat. No. 5,077,056). The loading is by pH gradient. It is preferable to begin with an internal pH of approximately pH 2-3. The excipient is the counterion in the loading process and when it comes in contact with the pharmaceutical agent in the interior of the liposome, the excipient may cause a substantial portion of the pharmaceutical agent to precipitate. The pharmaceutical agent may also self precipitate in the interior of the liposome. This precipitation protects the pharmaceutical agent and the lipids from degradation (e.g., hydrolysis). An excipient, such as citrate or sulfate, may precipitate the pharmaceutical agent and can be utilized in the interior of the liposomes together with a gradient (pH or ammonia) to promote drug loading.

Drug loading via the pH gradient includes a low pH in the internal aqueous space of the liposomes, and this internal acidity is, by design, incompletely neutralized during the drug loading process. This residual internal acidity can cause chemical instability in the liposomal preparation (e.g., lipid hydrolysis), leading to limitations in shelf life. To quench this residual internal acidity, membrane permeable bases, such as amines (e.g., ammonium salts or alkyl-amines) can be added following the loading of the pharmaceutical agent in an amount sufficient to reduce the residual internal acidity to a minimum value (for example, pH at or above 4). Ammonium salts that can be used include ones having mono- or multi-valent counterions, such as, but not limited to, ammonium sulfate, ammonium hydroxide ammonium acetate, ammonium chloride, ammonium phosphate, ammonium citrate, ammonium succinate, ammonium lactobionate, ammonium carbonate, ammonium tartrate, and ammonium oxalate. The analogous salt of any alkyl-amine compound which is membrane permeable can also be used, including, but not limited to, methylamine, ethylamine, diethylamine, ethylenediamine, and propylamine. During storage, for example at 2-8° C., the liposomal preparation will remain quenched, with reduced propensity for hydrolysis of either excipients or drug, relative to an un-quenched formulation. Upon injection, however, this quenching species rapidly leaks out of the liposome, thus restoring the residual gradient, which gradient is necessary for drug retention in vivo.

The therapeutic use of liposomes can include the delivery of drugs which are normally toxic in the free form. In the liposomal form, the toxic drug may be directed away from the sensitive tissue where toxicity can result and targeted to selected areas where they can exert their therapeutic effects. Liposomes can also be used therapeutically to release drugs slowly, over a prolonged period of time, thereby reducing the frequency of drug administration through an enhanced pharmacokinetic profile. In addition, liposomes can provide a method for forming an aqueous dispersion of hydrophobic drugs for intravenous delivery.

The route of delivery of liposomes can also affect their distribution in the body. Passive delivery of liposomes involves the use of various routes of administration, e.g., parenterally, although other effective administration forms, such as intraarticular injection, inhalant mists, orally active formulations, transdermal iotophoresis or suppositories are also envisioned. Each route produces differences in localization of the liposomes.

The invention also provides a method of preventing or treating inflammatory disease, by delivering a therapeutic or effective amount of liposomal composition of an inhibitor of MAP kinase, preferably in a mammal. Because dosage regimens for pharmaceutical agents are well known to medical practitioners, the amount of the liposomal pharmaceutical agent formulations which is effective or therapeutic for the treatment of the above mentioned diseases or conditions in mammals and particularly in humans will be apparent to those skilled in the art. The optimal quantity and spacing of individual dosages of the formulations herein will be determined by the nature and extent of the condition being treated, the form, route and site of administration, and the particular patient being treated, and such optimums can be determined by conventional techniques. It will also be appreciated by one of skill in the art that the optimal course of treatment, i.e., the number of doses given per day for a defined number of days, can be ascertained by those skilled in the art using conventional course of treatment determination tests.

The liposomes containing therapeutic agents (e.g., MAP kinase inhibitors or TNF-α antagonists) and the pharmaceutical formulations thereof of the present invention and those produced by the processes thereof can be used therapeutically in animals (including man) in the treatment of infections or conditions which require: (1) repeated administrations, (2) the sustained delivery of the drug in its bioactive form, or (3) the decreased toxicity with suitable efficacy compared with the free drug in question. Such conditions include but are not limited to inflammatory disease such as those that can be treated with MAP kinase inhibitors or TNF-α antagonists.

The mode of administration of the liposomes containing the pharmaceutical agents (e.g., MAP kinase inhibitors or TNF-α antagonists) and the pharmaceutical formulations thereof determine the sites and cells in the organism to which the compound will be delivered. The liposomes of the present invention can be administered alone but will generally be administered in admixture with a pharmaceutical carrier selected with regard to the intended route of administration and standard pharmaceutical practice. The preparations may be injected parenterally, for example, intravenously. For parenteral administration, they can be used, for example, in the form of a sterile aqueous solution which may contain other solutes, for example, enough salts or glucose to make the solution isotonic. The liposomes containing MAP kinase inhibitors or TNF-α antagonists, for example, may be given, as a 60 minute intravenous infusion at a dose of at least about 20 mg/m². They may also be employed for peritoneal lavage or intrathecal administration via injection. Other uses, depending on the particular properties of the preparation, may be envisioned by those skilled in the art.

For the oral mode of administration, the liposomal therapeutic drug (e.g., MAP kinase inhibitors or TNF-α antagonists) formulations of this invention can be used in the form of tablets, capsules; lozenges, troches, powders, syrups, elixirs, aqueous solutions and suspensions, and the like. In the case of tablets, carriers which can be used include lactose, sodium citrate and salts of phosphoric acid. Various disintegrants such as starch, and lubricating agents, such as magnesium stearate, sodium lauryl sulfate and talc, are commonly used in tablets. For oral administration in capsule form, useful diluents are lactose and high molecular weight polyethylene glycols. When aqueous suspensions are required for oral use, the active ingredient is combined with emulsifying and suspending agents. If desired, certain sweetening and/or flavoring agents can be added.

For the topical mode of administration, the liposomal therapeutic drug (e.g., MAP kinase inhibitors or TNF-α antagonists) formulations of the present invention may be incorporated into dosage forms such as gels, oils, emulsions, and the like. Such preparations may be administered by direct application as a cream, paste, ointment, gel, lotion or the like.

For administration to humans in the curative, remissive, retardive, or prophylactic treatment of inflammatory diseases, the prescribing physician will ultimately determine the appropriate dosage of the MAP kinase inhibitor drug or TNF-α antagonist drug for a given human subject, and this can be expected to vary according to the age, weight, and response of the individual as well as the nature and severity of the patient's disease. The dosage of the drug in liposomal form will generally be about that employed for the free drug. In some cases, however, it may be necessary to administer dosages outside these limits.

### Polymer Encapsulation

Biodegradable polyesters, such as poly(lactic acid) (PLA), poly(glycolic acid) (PGA), and poly(lactic-co-glycolic acid) (PLGA), have been extensively studied for a wide variety of pharmaceutical and biomedical applications. The biodegradable polyester family is a group of synthetic biodegradable polymers with controllable biodegradability, excellent biocompatibility, and high safety. The need for a variety of drug formulations for different drugs and delivery pathways has resulted in development of various types of block copolymers (e.g., diblock, triblock, multiblock, and star-shaped block) consisting of the biodegradable polyesters and poly(ethylene glycol) (PEG). Studies have demonstrated many desirable, unique properties of PLGA-PEG block copolymers. Synthesis of PLGA-PEG block copolymers are useful in applications such as drug delivery vehicles, micro/nano-particles, micelles, hydrogels, and injectable delivery systems. (Akina, Inc., W. Lafayette, Ind., www.akinainc.com/polycelle)

### Pharmaceutical Compositions and Methods of Administration

MAP kinase inhibitors, antagonists, anti-MAP kinase antibodies, TNF-α antagonists or anti-TNF-α antibodies can be used in treatment. In some methods, the genes encoding the inhibitors, antagonists, or antibodies are provided, such that the inhibitor, antagonist, or antibody bind to and modulate the MAP kinase or TNF-α protein within the cell. In other methods, a therapeutically effective amount of MAP kinase inhibitor or antagonist or TNF-α antagonist is administered to a patient. A “therapeutically effective amount”, “pharmacologically acceptable dose”, “pharmacologically acceptable amount” means that a sufficient amount of a MAP kinase inhibitor or antagonist, a TNF-α antagonist, or combination of agents is present to achieve a desired result, e.g., preventing, delaying, inhibiting or reversing a symptom of a disease or disorder or the progression of disease or disorder when administered in an appropriate regime.

Pharmaceutically acceptable carriers are determined in part by the particular composition being administered, as well as by the particular method used to administer the composition. Accordingly, there is a wide variety of suitable formulations of pharmaceutical compositions (see, e.g., Alfonso R Gennaro (ed), Remington: The Science and Practice of Pharmacy, (Formerly Remington's Pharmaceutical Sciences) 20th ed., Lippincott, Williams & Wilkins, 2003, incorporated herein by reference in its entirety). The pharmaceutical compositions generally comprise MAP kinase antagonist in a form suitable for administration to a patient. The pharmaceutical compositions are generally formulated as sterile, substantially isotonic and in full compliance with all Good Manufacturing Practice (GMP) regulations of the U.S. Food and Drug Administration.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, carriers known in the art.

In some methods, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like, particularly the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The nucleic acids, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the packaged nucleic acid with a base, including, for example, liquid triglycerides, polyethylene glycols, and paraffin hydrocarbons.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically or intrathecally. Formulations for injection can be presented in unit dosage form, e.g., in ampules or in multidose containers, with an added preservative.

Injection solutions and suspensions can be prepared from sterile powders, granules, and tablets of the kind previously described. Cells transduced by the packaged nucleic acid as described above in the context of ex vivo therapy can also be administered intravenously or parenterally as described above.

The dose administered to a patient should be sufficient to effect a beneficial therapeutic response in the patient over time. The dose will be determined by the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient.

In determining the effective amount of the vector to be administered in the treatment or prophylaxis of conditions resulting from expression of the MAP kinase proteins of the methods and compositions, the physician evaluates circulating plasma levels of the vector, vector toxicities, progression of the disease, and the production of anti-vector antibodies. In general, the dose equivalent of a naked nucleic acid from a vector is from about 1 μg to 100 μg for a typical 70 kilogram patient, and doses of vectors which include a retroviral particle are calculated to yield an equivalent amount of therapeutic nucleic acid.

For administration, inhibitors and transduced cells can be administered at a rate determined by the LD₅₀ of the inhibitor, vector, or transduced cell type, and the side-effects of the inhibitor, vector or cell type at various concentrations, as applied to the mass and overall health of the patient. Administration can be accomplished via single or divided doses.

Transduced cells are prepared for reinfusion according to established methods. See Abrahamsen et al., J. Clin. Apheresis 6:48-53, 1991; Carter et al., J. Clin. Arpheresis 4:113-117, 1998; Aebersold et al., J. Immunol. Meth. 112:1-7, 1998; Muul et al., J. Immunol. Methods, 101: 171-181, 1987; and Carter et al., Transfusion 27: 362-365, 1987, each incorporated herein by reference in their entirety. After a period of about 2-4 weeks in culture, the cells should number between 1×10⁸ and 1×10¹². In this regard, the growth characteristics of cells vary from patient to patient and from cell type to cell type. About 72 hours prior to reinfusion of the transduced cells, an aliquot is taken for analysis of phenotype, and percentage of cells expressing the therapeutic agent.

### Kits

The MAP kinase protein, antagonist or their homologs are useful tools for examining expression and regulation of signaling in nerve cells via the MAP kinase signaling pathway. Reagents that specifically hybridize to nucleic acids encoding MAP kinase proteins (including probes and primers of the proteins), and reagents that specifically bind to the proteins, e.g., antibodies, are used to examine expression and regulation.

Nucleic acid assays for the presence of MAP kinase proteins in a sample include numerous techniques are known to those skilled in the art, such as Southern analysis, northern analysis, dot blots, RNase protection, S1 analysis, amplification techniques such as PCR and LCR, high density oligonucleotide array analysis, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques 4:230-250, 1986; Haase et al., Methods in Virology, VII:189-226, 1984; and Nucleic Acid Hybridization: A Practical Approach (Hames et al., eds. 1987), each incorporated herein by reference in their entirety. In addition, MAP kinase protein can be detected with the various immunoassay techniques described above. The test sample is typically compared to both a positive control (e.g., a sample expressing recombinant MAP kinase protein) and a negative control.

Kits for screening nerve cell activity modulators. Such kits can be prepared from readily available materials and reagents are provided. For example, such kits can comprise any one or more of the following materials: the MAP kinase proteins, inhibitors, or antagonists, reaction tubes, and instructions for testing the activities of MAP kinase genes. A wide variety of kits and components can be prepared depending upon the intended user of the kit and the particular needs of the user. For example, the kit can be tailored for in vitro or in vivo assays for measuring the activity of MAP kinase proteins or nerve cell activity modulators.

Kits comprising probe arrays as described above are provided. Optional additional components of the kit include, for example, other restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions.

Usually, the kits also contain instructions for carrying out the methods.

Other embodiments and uses will be apparent to one skilled in the art in light of the present disclosures.

### EXEMPLARY EMBODIMENTS

### Example 1

### Phosphorylation of p38 MAPK in the Spinal Cord in Adjuvant Arthritis

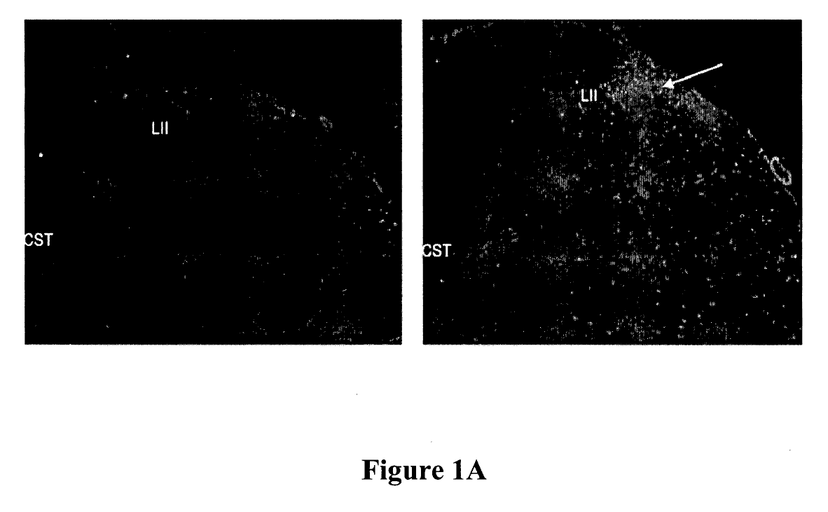

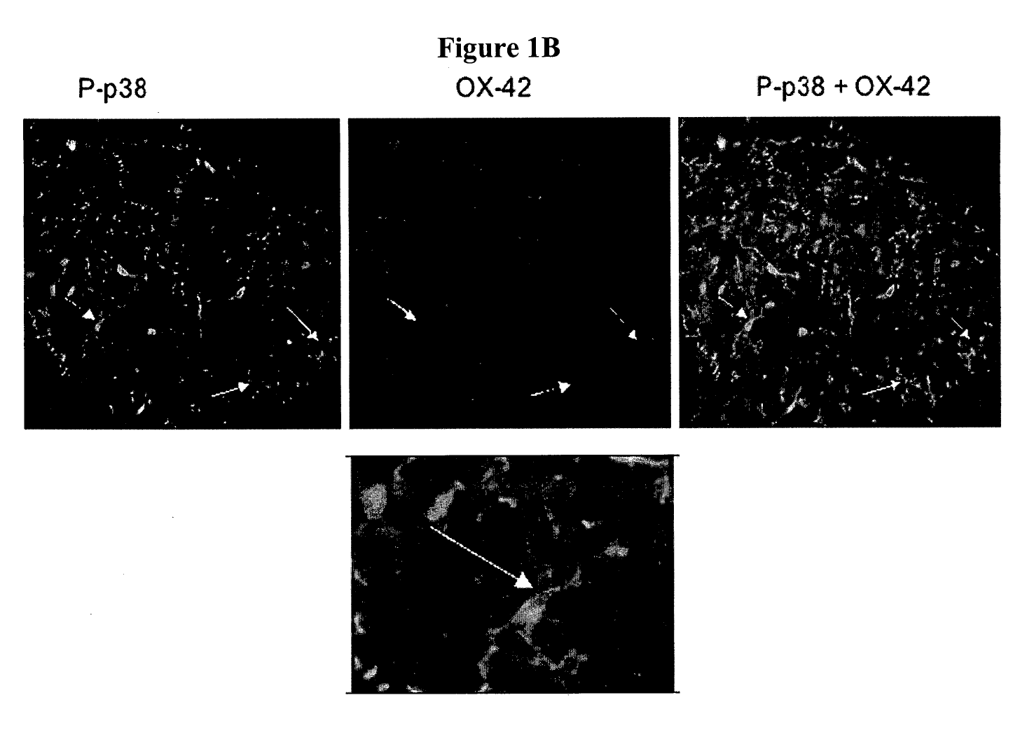

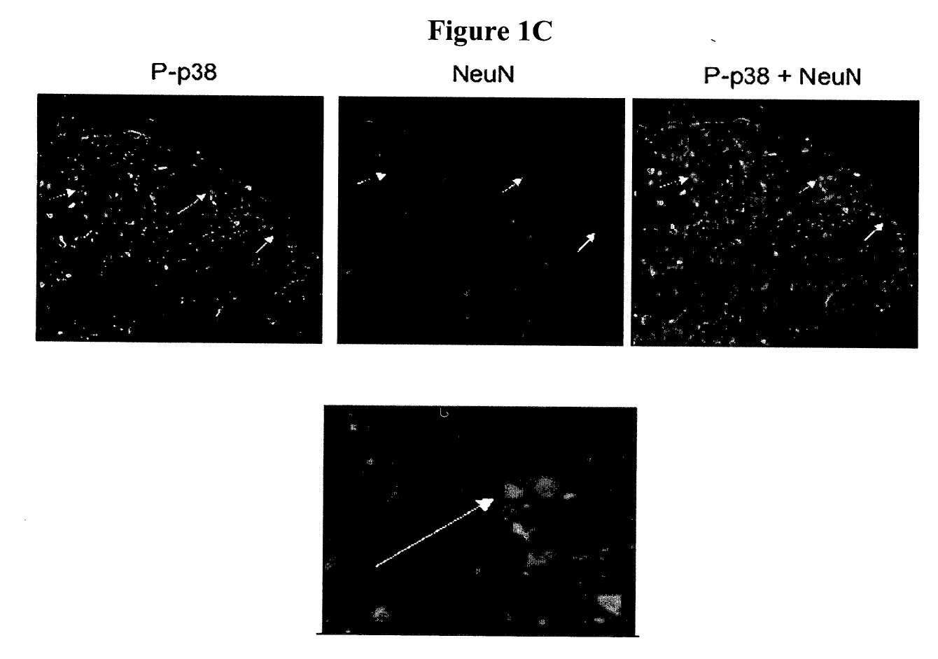

Rats were immunized with CFA on day 0 and sacrificed 1 week later. The L4/L5 spinal cord of immunized or control rats was harvested and tissue sections immunostained with FITC-anti-P-p38 antibody. Whereas dorsal horns of control rats contained only a few scattered P-p38 immunopositive cells, numerous cells stained positively for P-p38 in the CFA immunized rats (see FIG. 1A, right panel). These cells were present throughout the dorsal horn parenchyma, with especially dense staining in superficial laminae. To determine which cell types express P-p38 in arthritic animals, double staining was performed using anti-P-p38 and antibodies specific for activated microglial cells (OX-42) (FIG. 1B) or neurons (NeuN) (FIG. 1C). In the merged images (see FIG. 1B, top right panel and bottom right panel for a representative field), the majority of P-p38 was identified in microglial cells. Double staining studies also showed a limited number of neurons that expressed P-p38, and these were confined to lamina II (see FIG. 1C, top right panel and bottom right panel).

### Example 2

### Increased Phosphorylated P38 in the Spinal Cord in Adjuvant Arthritis

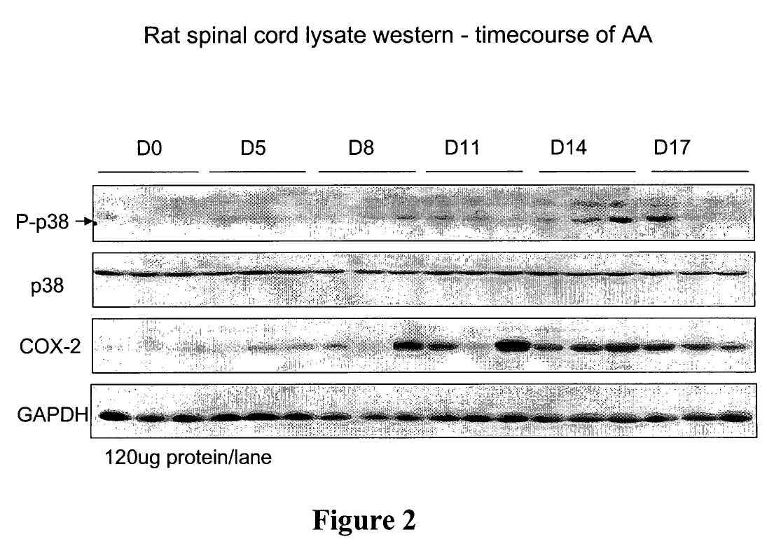

Rats were immunized with CFA on day 0 and sacrificed at various time points as indicated on FIG. 2 (Day 0 to Day 17). The L4/L5 spinal cord of immunized or control rats were harvested Western blots were performed. Note that phospho-p38 appears about 8 days after immunization (D8) and remains elevated until Day 17 (D17). Total p38 levels were unchanged in the spinal cord, and cyclo-oxygenase-2 (COX2) gene expression was also increased during the course of arthritis. GAPDH was used as a positive control for protein loading.

### Example 3

Intrathecal MAP Kinase Inhibitor Suppresses Adjuvant Arthritis

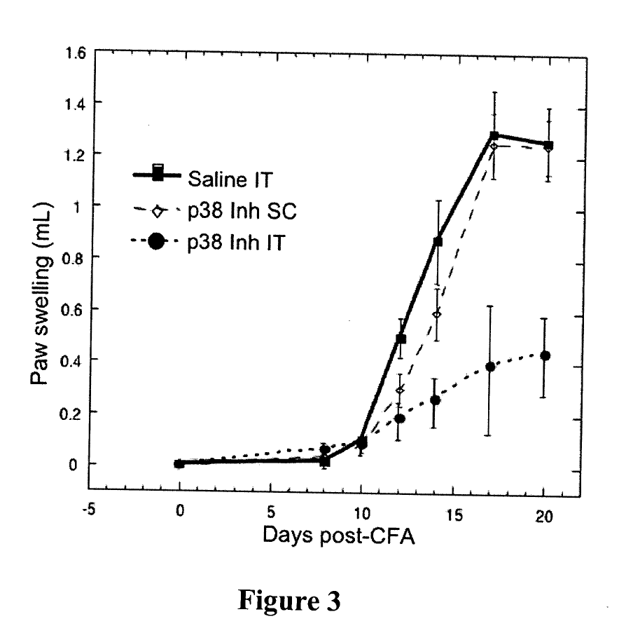

The effect of IT SB203580 on the clinical manifestations of adjuvant arthritis was examined. As shown in FIG. 3, paw swelling was significantly decreased in rats treated with IT SB203580 compared with either IT saline or the same dose of the inhibitor administered subcutaneously to catheterized rats (n=8/group). Paw swelling was inversely correlated with behavioral analysis as determined by weight bearing in hind limbs. Rats with IT drug treatment did not respond to ankle pressure with vocalization (pain response), in marked contrast to rats with either IT saline or systemic drug administration (Table 1).

TABLE 1
Behavioral pain effect of intrathecal p38 inhibitor in adjuvant arthritis

| Treatment | Positive vocalization* |
|---|---|
| IT Saline | 4/5 |
| IT SB203580 | 0/6** |
| SC SB203580 | 4/6 |
| No arthritis | 0/5 |

*24 gm bending force was applied to the lateral ankle of each hind paw for 4 s on day 20 after immunization. The number o animals that vocalize compared with the total number is shown.
**P < 0.05 compared with IT Saline (Chi square test).

FIG. 3 shows the effect of intrathecal p38 inhibitor on paw swelling in adjuvant arthritis. Rats were immunized with CFA on day 0 and treated daily with intrathecal SB203580 (8 μg), subcutaneous SB203580 (8 μg), or intrathecal saline beginning on day 8-20. Paw swelling was measured by water displacement plethysmometry. The intrathecal p38 inhibitor significantly decreased paw swelling.

### Example 4

### Intrathecal MAP Kinase Inhibitor Suppresses Joint Destruction

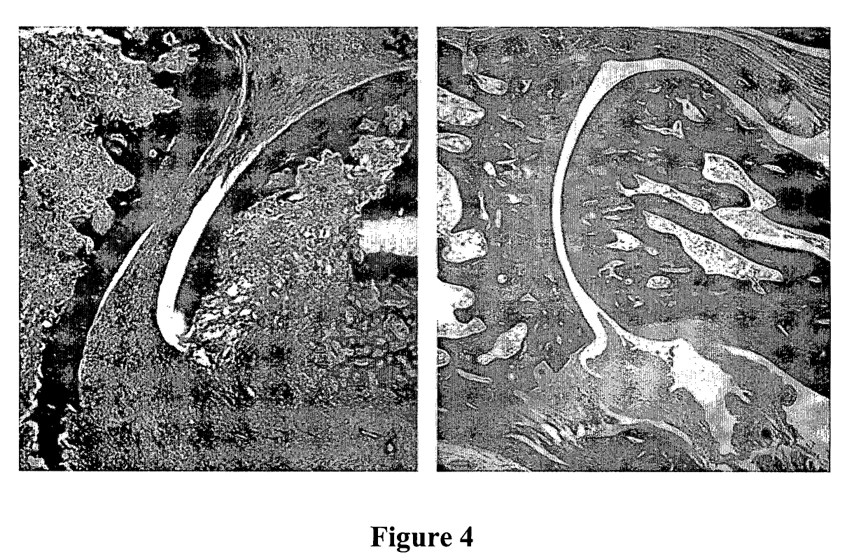

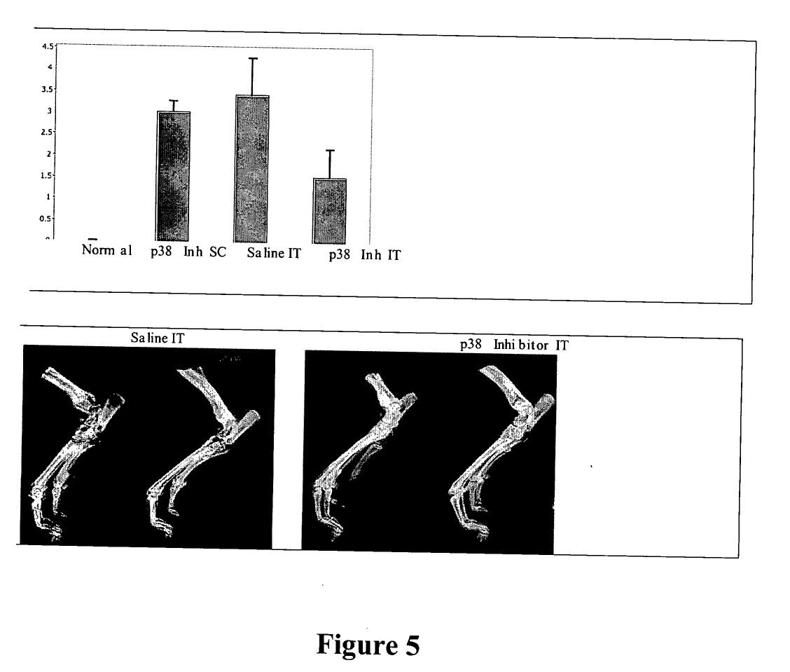

Histologic evaluation of the adjuvant arthritis model demonstrated lower synovial inflammation, bone erosion and cartilage loss in rats that receive IT SB203580 (see FIG. 4 for representative examples; Left panel is vehicle and right panel is SB203580). Using a semiquantitative scoring system, IT SB203580 significantly decreased joint damage and inflammation. Radiographic evidence of joint damage was also determined in the adjuvant arthritis model. Intrathecal (IT) treatment with the p38 inhibitor significantly decreased radiographic joint damage, including bone erosions, cartilage loss, and demineralization (p<0.05), while systemic treatment (subcutaneous, SC) with the same dose had no effect (see FIG. 5, top panel). Representative radiographs are also shown and demonstrate the protective effect of IT SB 203580 (see FIG. 5, bottom panel).

FIG. 4 shows the effect of intrathecal p38 inhibitor on histologic joint damage in adjuvant arthritis. Rats were immunized with CFA on day 0 and treated daily with intrathecal SB203580 (8 μg) or intrathecal saline beginning on day 8-20. Left panel is vehicle and right panel is SB203580. Hematoxylin and eosin stained sections were evaluated using a semiquantitative scoring system that demonstrated decreased damage in the SB203580-treated group. Representative sections are shown illustrating decreased erosions, cartilage damage and synovial inflammation in the treated group.

FIG. 5 shows the effect of intrathecal p38 inhibitor on radiographic joint damage in adjuvant arthritis. Rats were immunized with CFA on day 0 and treated daily with intrathecal SB203580 (8 μg), subcutaneous SB203580 (8 μg), or intrathecal saline beginning on day 8-20. The top panel shows the radiographic scores for normal, intrathecal p38 inhibitor (p38 Inh IT), intrathecal saline (saline IT), or p38 inhibitor injected subcutaneously (p38 Inh SC). Radiographs of the hind paws were evaluated using a semiquantitative scoring system that demonstrated decreased damage in the SB203580-treated group. Representative radiographs (bottom panel) are shown illustrating decreased joint destruction in the treated group (bottom panel left shows vehicle, bottom panel right shows IT SB203580).

### Example 5

Intrathecal MAP Kinase Inhibitor Suppresses Articular Cytokine and MMP Expression

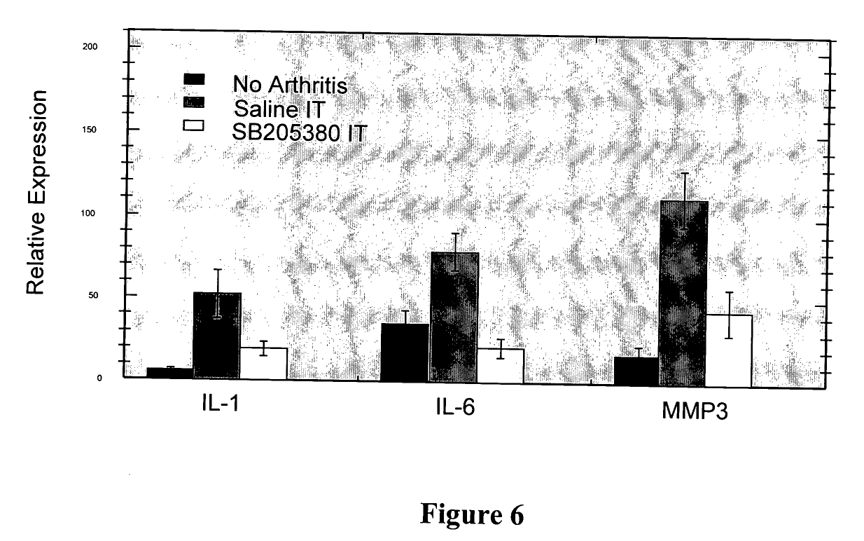

The effect of IT p38 inhibition on synovial pro-inflammatory gene expression was also determined using quantitative real time PCR. FIG. 6 shows that the IT p38 inhibitor decreased synovial IL-1 and IL-6. Furthermore, expression of a key gene involved with extracellular matrix production, MMP3, was also decreased (p<0.05). Normal rat synovium is also shown as a control.

FIG. 6 shows the effect of intrathecal p38 inhibitor on joint gene expression. Rats were immunized with CFA on day 0 and treated daily with intrathecal SB203580 (8 μg) or intrathecal saline beginning on day 8-20. (A) Quantitative real time PCR was performed on the hind paws. Normal rat joints are also shown as a control. Intrathecal SB203580 significantly decreased cytokine and MMP gene expression compared with intrathecal saline.

### Example 6

### Spinal Etanercept Decreases Peripheral Arthritis

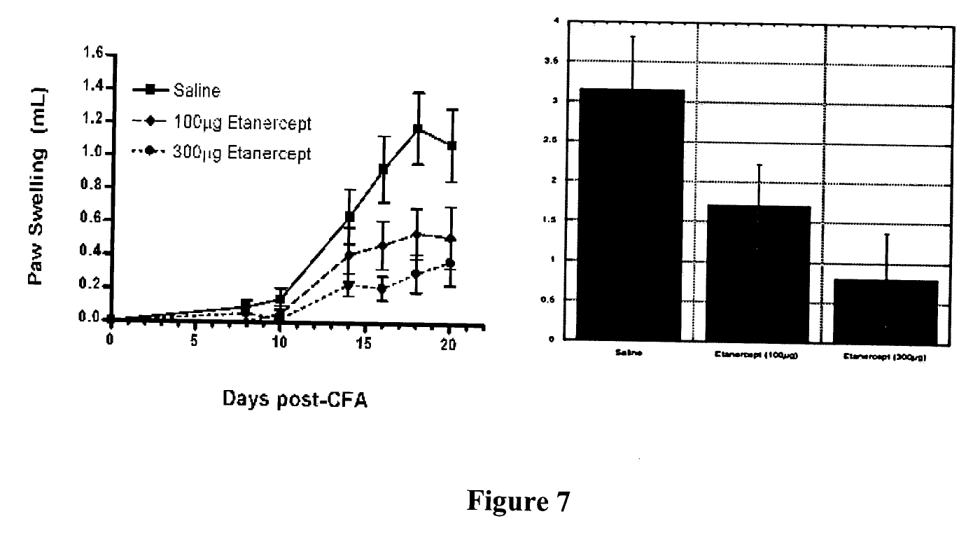

One possible mechanism of intrathecal p38 blockade is through the elaboration of spinal TNFα. In this case, we would anticipate that TNF blockade in the CNS would exhibit anti-inflammatory effects in adjuvant arthritis. To test this possibility, we evaluated treated rats immunized with CFA with 100 μg or 300 μg of etanercept IT beginning on day 0. As shown in FIG. 7, etanercept significantly decreased paw swelling (left panel) as well as radiographic evidence of joint destruction (right panel). Systemic treatment with the same doses of etanercept had no effect on paw swelling on day 20 (see Table 2).

TABLE 2
Systemic therapy with etanercept in adjuvant arthritis

| Treatment | Paw swelling (ml)* |
|---|---|
| Saline | 1.76 ± 0.22 |
| Etanercept 50 μg | 1.51 ± 0.20 |
| Etanercept 100 μg | 1.75 ± 0.18 |
| Etanercept 300 μg | 1.67 ± 0.16 |

Rats were immunized with CFA on day 0 and treated with saline or etanercept subcutaneously beginning on day 1 and continued every other day thereafter. Day 20 paw swelling is shown.

FIG. 7 shows the effect of intrathecal etanercept on adjuvant arthritis. Rats were immunized with CFA on day 0 and treated daily with intrathecal etanercept (100 or 300 μg q.o.d.) beginning on day 1 (N=6/group). A) Intrathecal etancercept significantly decreased paw swelling; and B) Intrathecal etanercept significantly decreases radiographic joint damage.

### Example 7

### Inflammation in Peripheral Tissues is Sensed by the Central Nervous System

Somatic afferent input transmission to the spinal cord from an inflammatory site can modulate the peripheral responses. However, the intracellular signaling mechanisms that regulate this linkage have not been defined. A novel spinal neural mechanism has been identified that controls peripheral inflammation through phosphorylation of spinal cord p38 mitogen activated kinase (MAPK). p38 in dorsal horn microglia and, to a lesser extent spinal cord neurons, is phosphorylated in rats with adjuvant arthritis, and kinase activity was increased as determined by in vitro kinase assay on spinal cord extracts. In an embodiment of the present invention, selective blockade of spinal cord p38 MAPK by administering the p38 inhibitor SB203580 via intrathecal catheters markedly suppressed paw swelling in adjuvant arthritis, decreased leukocyte infiltration into synovium, and decreased radiographic evidence of joint destruction. The same dose of SB203580 delivered systemically had no effect, indicating that the effect was mediated by local high concentrations in the central nervous system. Evaluation of articular gene expression by quantitative real time PCR showed that spinal p38 inhibition markedly decreased synovial cytokine (IL-1 and IL-6) and matrix metalloproteinase (MMP3) gene expression. These data suggest peripheral inflammation is sensed by the central nervous system, which subsequently activates stress-induced kinases in the spinal cord. Intracellular p38 MAP kinase signaling processes this information and through a feedback loop modulates somatic host responses. In an embodiment of the invention, this mechanism can have therapeutic implications and can contribute to the influence of the central nervous system on peripheral immune responses.

Neural networks in the CNS can influence somatic host responses in the peripheral tissues, but the mechanisms remain poorly defined. Previous studies described a neural reflex involving somatic afferent input that senses peripheral inflammation and implicate glutamate receptors in the spinal cord as part of a spinal circuit that modulates host responses in the skin and joints. In one aspect of the present invention, a novel intracellular mechanism has been identified that involves spinal p38 MAP kinase as one link between the periphery and the CNS. The surprising results indicate that selective spinal blockade of p38 MAPK has profound effects on peripheral inflammation, arthritis, and joint destruction.

To investigate spinal cord regulation of peripheral inflammation, the effect of intrathecal drug administration in a rat model of skin inflammation was examined. Rosengren, et al. J. Immunol. 154:5444-51, 1995. Adenosine (Ado) was selected as a potential central anti-inflammatory agent because hyperalgesia induced by inflammation is inhibited by adenosine A1 receptor agonists. Malmberg, et al. Anesthesiology 79:270-281, 1993. The analgesic action is mediated in part through inhibition of EAA release or by raising the NMDA receptor activation threshold. Dolphin, et al. Neurosci. Lett. 43:49-54, 1983; Corradetti, et al. Eur. J. Pharmacol. 104:19-26, 1984; Choca, et al. J. Pharmacol. Exp. Ther. 247:757-764, 1988; Schubert, et al. Acta Neurochirurgica [Suppl] 57:80-88, 1993. The studies on spinal cord regulation of inflammation demonstrated that spinal Ado receptor stimulation suppresses skin inflammation. Bong, et al. J Clin Invest 98:2779-2785, 1996 The action is independent of the sympathetic nerves as well as dopaminergic activity. Sorkin, et al. Exp. Neurol. 184:162-168, 2003. However, dorsal rhizotomy ablates the effect, indicating that afferent input and/or dorsal root reflexes are required for the anti-inflammatory activity. The mechanism also involves activation of A1 Ado receptors in the spinal cord, which subsequently decreases NMDA glutamate receptor function.

Other studies defining the influence of the CNS on peripheral inflammation have focused on vascular leakage rather than inflammatory cell recruitment. For instance, peripheral sympathectomy decreases paw swelling in adjuvant arthritis while it has no effect on peripheral neutrophil infiltration in carrageenan injected skin. Levine, et al. J. Neurosci. 6:3423-3429, 1986; Sluka, et al. Ann. Rheum. Dis. 53:309-14, 1994. Release of substance P release at the site of inflammation likely accounts for primary effects on vascular integrity rather than neutropihl trafficking. Green, et al. Neuroscience 55:1037-43, 1993; Khalil, et al. Clin. Exp. Neurol. 26:45-50, 1989; Cambridge, et al. Eur. J. Pharmacol. 301:151-7, 1996. Studies in intraarticular (knee) carrageenan-induced models demonstrate that spinal non-NMDA, rather than NMDA glutamate receptor subtypes mediate the edema. Sluka, et al. Pain 55:217-225, 1993. In contrast, neurogenic edema resulting from intradermal capsaicin injection is blocked by both non-NMDA and NMDA antagonists. Rees, et al. Neuroreport 5:821-824, 1994; Rees, et al. J. Physiol. 484:437-445, 1995; Lin, et al. J Neurophysiol. 82:2602-11, 1999. These contrasting results point to a high level of specificity of neural mediators with variations in type of inflamed tissue and inflammation.

While the glutamate receptors and neurotransmitters have been extensively evaluated in inflammation models, the role of spinal MAP kinases is much less clear. MAP kinases play a critical role in innate immunity and host defense by peripheral regulation of cytokines, like IL-1 and TNF-α, as well as enzymes involved in tissue remodeling, such as MMPs. Surprisingly, the same pathways are activated in spinal cord microglia and to a lesser extent in neurons when peripheral nerves are injured. Jin, et al. J. Neurosci. 23:4017-4022, 2003. For instance, P-p38 increases in spinal cord within a few hours after spinal nerve ligation, a standard model of neuropathic pain. Schafers et al. J Neurosci. 23:2517-21, 2003. p38 blockade by spinal administration of a p38 inhibitor substantially reduces sciatic nerve ligation-induced mechanical allodynia. After unilateral chronic constriction injury to the mid-sciatic nerve (another standard neuropathic pain model) or axotomy, P-p38 is elevated for up to 2 weeks after injury. Kim, et al. Neuroreport 13:2483-6, 2002. Increases in spinal ERK and JNK phosphorylation peak in dorsal horn astrocytes three weeks after partial sciatic nerve ligation. Ma et al. Pain. 99:175-84, 2002.

Data on activation of MAP kinases in the CNS due to inflammation are less clear. Carrageenan injection into rat footpads induces a 5-fold increase in spinal ERK phosphorylation. Galan, et al. Neurosci. Lett. 322:37-40, 2002. A similar increase in ERK activation occurs in an inflammatory pain model induced by injection of complete Freund's adjuvant (CFA) into rat paws. Obata, et al. J. Neurosci. 23:4117-26, 2003. Immunohistochemical studies show that both neurons and glia in the spinal cord contain the activated kinase. ERK activation after formalin injection appears to require metabotropic glutamate receptors rather than NMDA receptors. Karim, et al. J Neurosci. 21:3771-9, 2001. recent studies suggest that p38 can be activated in spinal cords by injection of carrageenan or formalin into the footpad and inhibition of p38 blocks pain behavior associated with the peripheral inflammation. Kim, et al. Neuroreport. 13:2483-6, 2002; Svensson et al. J. Neurochem. 86:1534-44, 2003. Another study reported that spinal administration of NMDA induced increases in P-p38 in superficial dorsal horn, predominantly in microglial, but also in a significant number of lamina II neurons, much the same pattern that as was observed in these studies. Svensson et al. Neuroreport. 14:1153-7, 2003.

Based on the studies demonstrating that nerve injury is associated with MAP kinase activation in the spinal cord and that p38 blockade has anti-hyperalgesic activity in animal models, these studies explored whether there is a corresponding effect on peripheral inflammation. These studies examined the activation state of p38 in the spinal cord during the evolution of chronic inflammation. Initial studies showed that p38 is phosphorylated in specific regions of the spinal cord dorsal horn 1 week after immunization. Both neurons and microglial cells in the spinal cord contain P-p38, and double staining studies suggest that the latter is the primary location. A therapeutic intervention with intrathecal SB203580, a commonly-used water soluble p38 inhibitor markedly decreased paw swelling and hyperalgesia. The amount required was only a small fraction of the dose required if the same compound was administered systemically, and the same low dose given subcutaneously had no effect in arthritic rats. More striking, inhibition of p38 in the CNS also decreased histologic evidence of synovial inflammation and suppressed radiographic signs of bone and cartilage destruction. Expression of pro-inflammatory genes and matrix metalloproteinases was also decreased by inhibition of p38 in the spinal cord.

Systemic p38 blockade has been extensively studied in inflammation and clearly demonstrates benefit in several models of arthritis. Nishikawa, et al. Arthritis Rheum. 48:2670-81, 2003; Jackson, et al. J. Pharmacol. Exp. Ther. 284:687-92, 1998; Badger, et al. J. Pharmacol. Exp. Ther. 279:1453-61, 1996. Of interest, the biological effects are similar to those observed in our studies evaluating IT administration of SB203580. Not only is synovial inflammation decreased, but also joint damage is markedly attenuated. One common observation is that very high doses of p38 inhibitors are often required when delivered by oral or parenteral routes. The effective dose in vivo is several hundred-fold higher than the amount required via IT therapy. These observations raise the intriguing possibility that the CNS might be responsible for a component of the anti-inflammatory effects seen with systemic administration of p38 inhibitors, and that inadequate CNS penetration accounts for this discrepancy.

The mechanism of central anti-inflammatory effects is likely related to the regulation of spinal or dorsal root gangion TNFα. Noxious stimuli in the periphery enhance spinal TNFα release, and etanercept, the TNFα antagonist, inhibits allodynia and p38 activation in spinal cord when administered intrathecally before spinal nerve ligation. Svensson et al. Spinal blockade of TNF blocks SNL-induced increases in spinal P-p38. Neurosci Lett, in press. Hence, TNFα can be both the result of and cause of p38 phosphorylation in neurons and microglia. To evaluate a central role of TNFα in adjuvant arthritis, we administered etanercept intrathecally in adjuvant arthritis. An earlier time point was selected for treatment (day 1 rather than day 8) because of the possibility that TNFα might induce p38 early in the model. Data of the present study show that spinal TNF blockade is as effect as its upstream regulator, p38, and that interceding at either point in the inflammatory cascade is sufficient to induce that peripheral anti-inflammatory effects.

The present studies have identified a novel connection between the CNS and peripheral inflammation that could have major implications for therapy. It is proposed that inflammation in peripheral tissues is sensed by the CNS, most likely through somatic afferent pathways, resulting in activation of p38 and perhaps other MAP kinases. This intracellular signaling pathway can subsequently relay information to the periphery, which is essential for full expression of somatic host responses. While it is possible that this efferent flow is via activation of dorsal root reflexes in the somatic nerves as has been shown for the vascular models, preliminary data indicate that mid thoracic spinal cord transection reduces the effect suggesting that there is also a supraspinal component. In addition to providing insights on the interactions between the CNS and host defense, this novel MAPK-dependent pathway could influence the design of MAPK inhibitors. The actions of p38 inhibitors might depend on CNS penetration for full anti-inflammatory and analgesic effects.

### Example 8

Material and Methods

Intrathecal (IT) catherization of rats. All animals were handled in accordance with USDA guidelines, and all procedures have been carefully reviewed and approved by the institutional animal subjects committee. Rats (200-300 g) were housed for at least one week before use. Isoflurane anesthesia was used for all surgical procedures. Animals were implanted with an intrathecal (IT) catheter modified from the method previously described. Yaksh, et al. Physiol. Behav. 17:1031-6, 1976. After a six day recovery period, all animals except those that appeared to have sensory or motor abnormalities (less than 5% of the total number) were used for experiments. For IT administration, 10 μl of drug or vehicle followed by 10 μl of isotonic saline was injected through the catheter.

Adjuvant arthritis. Male Lewis rats (150-200 g) were immunized at the base of the tail with 0.1 ml of complete Freund's adjuvant (CFA) on day 0. Drug (IT or subcutaneous SB203580 or IT vehicle) treatment started on day 8 and continued on a daily basis until day 20. Clinical signs of arthritis generally begin on day 10, and paw swelling was determined every second day by water displacement plethysmometry. Power analysis indicates that 8-10 animals per group will detect a 30% decrease in severity of arthritis. Prior to daily drug delivery, rats were individually assessed for ability to walk and bear weight on their hind paws. On the day of sacrifice, a probe with a 24 gm bending force was applied to the lateral ankle of each hind paw for 4 s. Animals were graded (0/1) on whether they vocalized. Roentgenograms were obtained of the hind paws to assess bone changes using a semi-quantitative scoring system (demineralization (0 to 2+), ankle and mid-foot erosions (0 to 2+), calcaneal erosion (0 to 1+), heterotopic bone formation (0 to 1+) (maximum possible score=6). Paws were fixed in formalin and decalcified for 2-3 days. Sections from paraffin embedded tissue were stained with hematoxylin and eosin. A synovial inflammation score was determined using a semi-quantitative scale that measures synovial inflammation (0-2+), cartilage integrity (0-2+), bone erosions (0-2+), marrow infiltration (0-2+), proteoglycans loss (0-2+ in safranin 0 stained sections) and extra-articular inflammation (0-2+) (maximum score 12). Subjective assessments were made by an investigator blinded to the drug treatment.

Immunostaining of spinal cords. Animals were anesthetized with 4% isoflurane and perfused first with room temperature 0.9% heparinized saline containing phosphatase inhibitors followed by 4% chilled paraformaldehyde/0.1 M phosphate buffer. Spinal cords were removed, and the fixed tissue was mounted in Tissue-Tek ornithine transcarbamylase embedding compound, snap frozen, and stored at −80° C. 10 μm sections were thaw-mounted onto Superfrost Plus Slides and air-dried for 30 min. Sections were postfixed in acetone at 20° C. and blocked with 10% BSA/PBS. Serial sections and/or double staining of the same section were used to determine co-localization of P-p38 with cell markers (Ox42 for microglia and NeuN for neurons). Sections were incubated at 4° C. for 24 hr and washed three times for 10 min each in PBS. An ABC system (Vector Laboratories, Burlingame, Calif.) with Cy3-, Cy2-, or FITC conjugated secondary Ab were used for detection.

Immunoprecipitation and in vitro kinase reactions. Tissue was homogenized in lysis buffer and insoluble material removed by centrifugation at 22,000×g for 15 minutes at 4° C. Supernatant containing 250 μg of protein in a total volume of 100 μl was incubated with anti-p38 antibody. Then 30 μl of 50% slurry of protein A-Sepharose 4B-CL in PBS was then added and the mixture incubated for 1 h on a rotation wheel. After centrifugation for 5 min at 3,000×g, immunocomplexes were washed 3 times with low-salt buffer and 3 times with a high-salt buffer and once with 20 mM HEPES, pH 7.4, 25 mM MgCl2. The kinase reaction was started by adding 30 μl of kinase buffer, 2 μCi of γ[32P]-ATP) and 3 μg of substrate for 30 min at 37° C. Laemmli loading buffer was added, samples heated for 5 min at 95° C., separated using 10% SDS-PAGE, and visualized by autoradiography.

Q-PCR. Ankle joints were excised and trimmed of skin. The tissue was then pulverized and RNA extracted using RNA-STAT-60 and standard techniques. The RNA was reversed transcribed into cDNA using random hexamer primers. TaqMan PCR amplification reactions were carried out in reaction volumes of 25 μl in MicroAmp Optical Plates and MicroAmp Optical Caps using the TaqMan Universal Master Mix with a Perkin Elmer 5700 thermal cycler. Each sample was analyzed in triplicate using 50 ng of cDNA in the reaction. Thermal cycling was initiated with a 2 min incubation at 50° C., followed by 10 min at 95° C. to activate the AmpliTaq polymerase and then 40 cycles of 95° C. for 15 s/60° C. for 1 min. GADPH was used as a loading control for in each sample. The standard curve method was used for quantification as previously described. Boyle, et al. Arthritis Res. Ther. 5:R352-360, 2003.

When ranges are used herein for physical properties, such as molecular weight, or chemical properties, such as chemical formulae, all combinations and subcombinations of ranges and specific embodiments therein are intended to be included.

The disclosures of each patent, patent application and publication cited or described in this document are hereby incorporated herein by reference in their entirety.

Those skilled in the art will appreciate that numerous changes and modifications can be made to the embodiments of the invention and that such changes and modifications can be made without departing from the spirit of the invention. It is, therefore, intended that the appended claims cover all such equivalent variations as fall within the true spirit and scope of the invention.

What is claimed:

1. A method for preventing or treating inflammatory disease in a mammal comprising administering an inhibitor of mitogen activated (MAP) kinase targeted to the central nervous system of said mammal in a therapeutic amount to said mammal in need thereof.

2. The method of claim 1 wherein the MAP kinase is p38 MAP kinase, p38α MAP kinase, or p3813 MAP kinase.

3. The method of claim 1 wherein the inflammatory disease is peripheral inflammation, acute inflammation, chronic inflammation, arthritis, or rheumatoid arthritis.

4. The method of claim 1, wherein the inflammatory disease is bone resorption, graft vs. host reaction, atherosclerosis, arthritis, osteoarthritis, rheumatoid arthritis, gout, psoriasis, topical inflammatory disorder state, adult respiratory distress syndrome, asthma, chronic pulmonary inflammatory disorder, cardiac reperfusion injury, renal reperfusion injury, thrombus, glomerulonephritis, Crohn's disorder, ulcerative colitis, inflammatory bowel disorder, or cachexia.

5. The method of claim 1, wherein the MAP kinase inhibitor is an antisense oligonucleotide to p38 MAP kinase, p38α MAP kinase, or p38β MAP kinase.

6. The method of claim 1, wherein the MAP kinase inhibitor is an interfering RNA to p38 MAP kinase, p38α MAP kinase, or p38β MAP kinase.

7. The method of claim 2, wherein the p 38 MAP kinase inhibitor is SB023580.

8. The method of claim 1, wherein the MAP kinase is JNK or MEK1/2.

9. The method of claim 8, wherein the MAP kinase inhibitor is SP600125 or PD98059.

10. The method of claim 1, wherein the MAP kinase inhibitor is administered intrathecally, intramedullarly, intracerebrally, intracerebroventricularly, intracranially, epidurally, intraspinally, or intraparietally.

11. The method of claim 10, wherein the MAP kinase inhibitor crosses the blood-brain barrier of the mammal.

12. The method of claim 1, wherein the MAP kinase inhibitor is administered intranasally to the mammal.

13. The method of claim 12, wherein the MAP kinase inhibitor crosses the blood-brain barrier of the mammal.

14. The method of claim 1, wherein the MAP kinase inhibitor is administered systemically.

15. The method of claim 1, wherein the MAP kinase inhibitor is administered intravenously, parenterally, subcutaneously, intramuscularly, ophthalmicly, intraventricularly, intraperitoneally, orally, or topically, to said mammal.

16. The method of claim 1, wherein an encapsulated form of the MAP kinase inhibitor is administered in a lipophilic compound or liposome.

17. The method of claim 10, wherein an encapsulated form of the MAP kinase inhibitor is administered intrathecally into the cerebrospinal fluid of the subject at an entry region.

18. The method of claim 17 wherein the entry region is not a lumbar region.

19. The method of claim 10, wherein the MAP kinase inhibitor is encapsulated in a polymer.

20. The method of claim 19, wherein the polymer is a naturally derived polymer, albumin, alginate, cellulose derivatives, collagen, fibrin, gelatin, or polysaccharide.

21. The method of claim 19, wherein the polymer is a synthetic polymer, polyester, polylactic acid, polyglycolic acid, polylactic-co-glycolic acid, polyethylene glycol, poloxomer block copolymer, or polyanhydride.

22. The method of claim 19, wherein an encapsulated form of the therapeutic agent is introduced into a cerebral ventricle.

23. The method of claim 19, wherein an encapsulated form of the therapeutic agent is introduced into the cisterna magna.

24. The method of claim 19, wherein an encapsulated form of the therapeutic agent is introduced into the lumbar region.

25. The method of claim 17, wherein the MAP kinase inhibitor is administered intrathecally to a sacral region of the central nervous system of the subject.

26. A method of treating, reducing, or preventing inflammation comprising contacting the periphery of a mammal with a compound that decreases the enzymatic activity or phosphorylation level of a MAP kinase in the central nervous system of the mammal, in an amount sufficient to treat, reduce, or prevent inflammation.

27. The method of claim 26, wherein said compound is administered intrathecally, intramedullarly, intracerebrally, intracerebroventricularly, intracranially, epidurally, intraspinally, or intraparietally.

28. The method of claim 26, wherein said compound crosses the blood-brain barrier of said mammal.

29. The method of claim 26, wherein said compound is administered systemically.

30. The method of claim 29, wherein said contacting comprises administering said compound intravenously, parenterally, subcutaneously, intramuscularly, ophthalmicly, intraventricularly, intraperitoneally, orally, topically, or intranasally to said mammal.

31. The method of claim 26, wherein an encapsulated form of the MAP kinase inhibitor is administered in a lipophilic compound or liposome.

32. The method of claim 26, wherein the MAP kinase inhibitor is encapsulated in a polymer.

33. A method for preventing or treating inflammatory disease in a mammal comprising administering an antagonist of TNF-α targeted to the central nervous system of said mammal in a therapeutic amount to said mammal in need thereof.

34. The method of claim 33 wherein the inflammatory disease is peripheral inflammation, acute inflammation, chronic inflammation, arthritis, or rheumatoid arthritis.

35. The method of claim 33, wherein the inflammatory disease is bone resorption, graft vs. host reaction, atherosclerosis, arthritis, osteoarthritis, rheumatoid arthritis, gout, psoriasis, topical inflammatory disorder state, adult respiratory distress syndrome, asthma, chronic pulmonary inflammatory disorder, cardiac reperfusion injury, renal reperfusion injury, thrombus, glomerulonephritis, Crohn's disorder, ulcerative colitis, inflammatory bowel disorder, or cachexia.

36. The method of claim 33, wherein the TNF-α antagonist is an antisense oligonucleotide.

37. The method of claim 33, wherein the TNF-α antagonist is an interfering RNA.

38. The method of claim 34, wherein the TNF-α antagonist is etanercept, infliximab, or adalimumab.

39. The method of claim 33, wherein the TNF-α antagonist is administered intrathecally, intramedullarly, intracerebrally, intracerebroventricularly, intracranially, epidurally, intraspinally, or intraparietally.

40. The method of claim 39, wherein TNF-α antagonist crosses the blood-brain barrier of the mammal.

41. The method of claim 33, wherein the TNF-α antagonist is administered intranasally to the mammal.

42. The method of claim 41, wherein the TNF-α antagonist crosses the blood-brain barrier of the mammal.

43. The method of claim 33, wherein the TNF-α antagonist is administered systemically.

44. The method of claim 33, wherein the TNF-α antagonist is administered intravenously, parenterally, subcutaneously, intramuscularly, ophthalmicly, intraventricularly, intraperitoneally, orally, or topically, to said mammal.

45. The method of claim 33, wherein an encapsulated form of the TNF-α antagonist is administered in a lipophilic compound or liposome.

46. The method of claim 39, wherein an encapsulated form of the TNF-α antagonist is administered intrathecally into the cerebrospinal fluid of the subject at an entry region.

47. The method of claim 46 wherein the entry region is not a lumbar region.

48. The method of claim 39, wherein the TNF-α antagonist is encapsulated in a polymer.

49. The method of claim 48, wherein the polymer is a naturally derived polymer, albumin, alginate, cellulose derivatives, collagen, fibrin, gelatin, or polysaccharide.

50. The method of claim 48, wherein the polymer is a synthetic polymer, polyester, polylactic acid, polyglycolic acid, polylactic-co-glycolic acid, polyethylene glycol, poloxomer block copolymer, or polyanhydride.

51. The method of claim 48, wherein an encapsulated form of the therapeutic agent is introduced into a cerebral ventricle.

52. The method of claim 48, wherein an encapsulated form of the therapeutic agent is introduced into the cisterna magna.

53. The method of claim 48, wherein an encapsulated form of the therapeutic agent is introduced into the lumbar region.

54. The method of claim 46, wherein the TNF-α antagonist is administered intrathecally to a sacral region of the central nervous system of the subject.

55. A method of treating, reducing, or preventing inflammation comprising contacting the periphery of a mammal with a compound that decreases the activity of TNF-α in the central nervous system of the mammal, in an amount sufficient to treat, reduce, or prevent inflammation.

56. The method of claim 55, wherein said compound is administered intrathecally, intramedullarly, intracerebrally, intracerebroventricularly, intracranially, epidurally, intraspinally, or intraparietally.

57. The method of claim 55, wherein said compound crosses the blood-brain barrier of said mammal.

58. The method of claim 55, wherein said compound is administered systemically.

59. The method of claim 58, wherein said contacting comprises administering said compound intravenously, parenterally, subcutaneously, intramuscularly, ophthalmicly, intraventricularly, intraperitoneally, orally, topically, or intranasally to said mammal.

60. The method of claim 55, wherein the TNF-α antagonist is administered in an encapsulated form in a lipophilic compound or liposome.

61. The method of claim 55, wherein the TNF-α antagonist is encapsulated in a polymer.
